(19) United States
(12) Patent Application Publication
(10) Pub. No.: US 2026/0234617 A1
(43) Pub. Date: Aug. 13, 2026

(54) PHARMACEUTICAL FORMULATIONS OF GENE DELIVERY VEHICLES

(71) Applicant: UNIQURE BIOPHARMA B.V., Amsterdam (NL)

(72) Inventors: Maria Mihaela APETRI, Amsterdam (NL); Gabriel FAIMAN, Amsterdam (NL); Edina LJUBOVIC-COUTEAU, Amsterdam (NL); Simi MATHEW, Amsterdam (NL)

(73) Assignee: UNIQURE BIOPHARMA B.V., Amsterdam (NL)

(21) Appl. No.: 19/148,036

(22) PCT Filed: Jan. 19, 2024
(86) PCT No.: PCT/EP2024/051203

§ 371 Date: Jul. 15, 2025

(30) Foreign Application Priority Data
Jan. 19, 2023 (EP) 23152369.7

(51) Int. Cl. C12N 15/113; A61K 35/76

(52) U.S. Cl. CPC C12N 15/113; A61K 35/76; C12N 2310/141

(57) ABSTRACT

The invention relates to formulations comprising miRNA that have improved stability for the treatment of diseases including neurodegenerative diseases such as spinocerebellar ataxias type 3.

### TECHNICAL FIELD

The invention relates to the field of gene therapy. In addition, the invention relates to the field of interfering RNA and/or microRNA (miRNA). In particular, the invention relates to gene therapy involving such miRNA and more in particular to pharmaceutical formulations with improved stability for the treatment of diseases including neurodegenerative diseases such as spinocerebellar ataxias type 3.

### Gene Therapy

The elucidation of DNA as the carrier of genetic information, and therefore also as the source of inherited diseases, has led to envisaged therapies in which mutant, damaged genes could be replaced or at least silenced. Many genes and/or other nucleic acid sequences have now been identified to play a role in (genetic) disease. If the mutant gene(s) could be replaced by a healthy one, or if the genes expressing aberrant (sometimes toxic) products could be silenced, the disease could be treated at the molecular level and potentially be cured. Gene therapy provides a promising concept, in particular for diseases caused by mutations in a single gene.

However, the delivery of the desired nucleic acid to the cells that need to be targeted is not an easy task. Numerous (viral) delivery systems have been investigated, all of them with their advantages and drawbacks. One of the viral delivery vehicles that are used for gene therapy is the adeno-associated virus (AAV).

### AAV

AAV has a single-stranded DNA genome of approximately 4.8 kilobases (kb). AAV belongs to the parvovirus family and is dependent for replication on co-infection with other viruses, in particular adenoviruses. The genome comprises Rep (Replication) and Cap (Capsid) genes. These coding sequences are flanked by inverted terminal repeats (ITRs) that are required for genome replication and packaging. The Rep gene encodes four proteins (Rep78, Rep68, Rep52, and Rep40), replicates the viral genome, and facilitates packaging, while Cap expression gives rise to the viral capsid proteins (VP; VP1 VP2 VP3), which form the outer capsid shell.

For gene therapy, the viral DNA of the AAV is almost completely removed. Recombinant AAV (rAAV) for gene therapy is formed by a protein capsid containing a desired nucleic acid, the transgene, that is to be delivered to target cells. The desired nucleic acid is flanked by the ITR's of AAV. ITR-flanked transgenes encoded by rAAV can form circular concatemers remaining in the nucleus of transduced cells as episomes. As the episome remains largely episomal, the expression of AAV delivered nucleic acid sequences may be diluted over time if and when the target cell replicates. This dilution may not generally apply to post-mitotic cells such as neurons, which are the target cells for many neurodegenerative diseases. A review on AAV vectors for gene therapy is provided in Naso et al, Biodrugs 2017 (p. 317-334).

miRNA

RNA interference (RNAi) is a naturally occurring mechanism that involves sequence-specific down-regulation of messenger RNA (mRNA). The down-regulation of mRNA results in a reduction of the amount of protein that is expressed. RNA interference is triggered by double-stranded RNA. One of the strands of the double-stranded RNA is substantially or completely complementary to its target, the mRNA. This strand is termed the guide strand. The mechanism of RNA interference involves the incorporation of the guide strand in the RNA-induced silencing complex (RISC). This complex is a multiple turnover complex that via complementary base paring of the guide strand can bind to its target mRNA. Once bound to its target mRNA it can either cleave the mRNA or reduce translation efficiency. RNA interference has since its discovery been widely used to knock down specific target genes. The triggers for inducing RNA interference that have been employed involve the use of small interfering RNAs (siRNAs) or short hairpin RNAs (shRNAs). In addition, molecules that can naturally trigger RNAi, the so-called microRNAs (miRNAs), have been used to make artificial (engineered) miRNAs that mimic their naturally occurring counterparts. These strategies have in common that they provide for substantially double-stranded RNA molecules that are designed to target an mRNA of choice. RNAi-based therapeutic approaches that utilise the sequence-specific modality of RNAi are under development and several are currently in clinical trials (see i.a. Davidson and McCray, Nature Reviews—Genetics, 2011; Vol. 12; 329-340).

### Spinocerebellar Ataxia Type 3 (SCA3)

Spinocerebellar ataxia type 3 (SCA3), or Machado-Joseph disease (MJD), is an autosomal dominant monogenic, fatal disorder. The disorder is characterized by progressive degeneration of brain areas, which is caused by a CAG expansion in the human ataxin-3 gene, also referred to as the ATXN3 gene (OMIM: 607047, reference sequence Homo sapiens ataxin 3 (ATXN3) on chromosome 14, NCBI Reference Sequence: NG_008198.2). In the 3′ regions of the gene, a cytosine-adenine-guanine (CAG) repeat region is present. Said CAG region is in the frame and results in an ataxin-3 protein comprising a polyQ region, a repetitive sequence of glutamines. Healthy, or non-symptomatic, individuals may have up to 44 CAG-repeats in the ATXN3 gene. Diseased individuals have expansions and it has been shown that they may have between 52 and 86 or more CAG repeats. Individuals having between 45-51 CAG repeats are to have symptoms with incomplete penetrance of the disease. Said expansion results in an ataxin-3 protein that has extended polyQ regions and the length of the CAG repeats, and thus polyQ regions within ataxin-3, can be correlated with disease progression, i.e. the longer the region usually the more progressive the disease.

The ataxin-3 protein with the expanded polyQ tract acquires toxic properties (gain of toxic function) and the formation of neuronal aggregates in the brain is the neuropathological hallmark. Neuropathological studies have detected a widespread neuronal loss in various areas, including the cerebellum, thalamus, midbrain, pons, medulla oblongata, and spinal cord of SCA3 patients (Riess et al., Cerebellum 2008). Although widespread pathology is reported, the consensus is that the main pathology is in the cerebellum and the brainstem (Eichler et al. AJNR Am J Neuroradiol, 2011). The disease has full penetration, which means that if a person has an expansion of 52 or more CAGs, they will inevitably develop the disease and have a 50% chance to pass it on to their offspring.

### Formulation and Administration Challenges

Using AAV vectors to deliver micro-RNAs directly to a patient's brain for knockdown of the ATXN3 gene could represent a highly innovative and promising approach for treating SCA3.

However, administration directly to a patient's brain or spine presents special challenges for the formulation and stability of a suitable pharmaceutical formulation, such as the need to avoid undesirable formation of aggregated or agglomerated particles in the formulation that may be detrimental to a patient. For example, such formulations may be stored frozen and then thawed before use. Aggregates or agglomerates could problematically form during or after the thaw of the formulation, and also during the time following thawing but before administration of the formulation. It would therefore be highly desirable to provide an innovative formulation of an AAV vector for treating spinocerebellar ataxia type 3, or other diseases, that eliminates or substantially eliminates aggregate or agglomerate formation for an extended in-use shelf life.

### SUMMARY OF INVENTION

In a first aspect there is provided an isotonic pharmaceutical formulation comprising:
a buffer
an adeno-associated viral vector with a transgene encoding a microRNA;
a tonicity agent;
a cryoprotectant; and
a surfactant.

In some embodiments, the formulation has an osmolarity of from 250 to 330 mOsm/kg and a pH value of from 5 to 8.

In some embodiments, the formulation is substantially free of visible particles.

In some embodiments, the adeno-associated viral vector with a transgene encoding microRNA targets ATXN mRNA, for example, ATXN3 mRNA.

In some embodiments, the adeno-associated viral vector comprises an AAV2 serotype, an AAV5 serotype, an AAV9 serotype, a hybrid AAV serotype, or a combination thereof, preferably the adeno-associated viral vector comprises an AAV5 serotype or an AAV9 serotype, more preferably, an AAV5 serotype.

In some embodiments, the concentration of the adeno-associated viral vector is at least 1E13 gc/ml, such as at least 3E13 gc/ml. In some embodiments, the concentration of the adeno-associated viral vector is between 0.5E13 and 6E13 gc/ml, such as between 1E13 and 5E13 gc/ml.

In some embodiments, the cryoprotectant is a carbohydrate selected from the group consisting of trehalose, sucrose, dextrose, dextran, and combinations thereof; and present in an amount of up to 8% w/v.

In some embodiments, the tonicity agent is selected from the group consisting of NaCl, KCl, MgCl₂, CaCl₂, and combinations thereof; and is present in an amount of from 5 mM to 150 mM. In some embodiments, the surfactant is a non-ionic surfactant; and is present in an amount of from 0.001% v/v to 0.1% v/v, preferably from 0.003% v/v to 0.08% v/v, more preferably from 0.005% v/v to 0.02% v/v, for example about 0.01% v/v.

In some embodiments, the buffer is selected from the group consisting of an artificial cerebrospinal fluid (aCSF), acetate buffer, citrate buffer, phosphate buffer, HEPES, Tris, and combinations thereof.

In some embodiments, the formulation remains stable at room temperature (15° C.-25° C.) without substantial formation of aggregates or agglomerates for at least 12 hours after thawing.

In a second aspect, there is provided the formulation of the invention for use in treating Spinocerebellar Ataxia (SCA).

In some embodiments, the formulation is administered by intra-CSF (cerebral spinal fluid) delivery.

In some embodiments, the formulation is administered to a patient's cisterna magna.

### DETAILED DESCRIPTION OF INVENTION

The present invention seeks to provide a new pharmaceutical formulation that has reduced aggregate or agglomerate formation during processing and is essentially free of visible particles for an extended time at room temperature after thawing. The present invention also seeks to provide a pharmaceutical formulation comprising one or more excipients that is substantially isotonic to human cerebrospinal fluid (CSF) for improved administration to a patient's brain. The present invention also seeks to provide a method of treating spinocerebellar ataxia type 3 (SCA3) by administering the formulation of the invention to a patient's brain.

In one aspect of the invention, the present invention provides an isotonic pharmaceutical formulation comprising a buffer, an adeno-associated viral vector with a transgene encoding a microRNA, a tonicity agent, a cryoprotectant, and a surfactant.

In this way, it has been surprisingly found that the addition of a cryoprotectant and a surfactant to the formulation helps to improve the stability of the drug product, in particular under high concentration. The formulation may remain stable without substantial formation of aggregates or agglomerates for at least about 6 months, preferably at least about 12 months, more preferably at least about 24 months, for example at least about 36 months, at a storage temperature of ≤−65° C. The formulation may also remain stable at room temperature (15° C.-25° C.) without substantial formation of aggregates or agglomerates for an extended time, preferably at least about 12 hours, more preferably at least about 24 hours, after thawing.

In some embodiments, the adeno-associated viral vector with a transgene encoding a microRNA may target ATXN mRNA, which may enable the formulation to treat spinocerebellar ataxia. For example, the adeno-associated viral vector with a transgene encoding a microRNA may target ATXN3, thereby lowering ATXN3 mRNA and protein levels in the treatment of spinocerebellar ataxia type 3. Therefore, in some embodiments, the adeno-associated viral vector with a transgene encoding microRNA targets ATXN mRNA. In further embodiments, the adeno-associated viral vector with a transgene encoding microRNA targets ATXN3 mRNA.

In some embodiments, the adeno-associated viral vector comprises an AAV2 serotype, an AAV5 serotype, an AAV9 serotype, a hybrid AAV serotype, or a combination thereof. Said serotypes have been found particularly preferred for the treatment of spinocerebellar ataxia type 3. In some embodiments, the adeno-associated viral vector comprises an AAV9 serotype.

In some embodiments, the adeno-associated viral vector comprises an AAV5 serotype.

In some embodiments, the adeno-associated viral vector comprises a hybrid AAV serotype. By way of example, the hybrid AAV serotype may be a hybrid AAV2/AAV5; AAV2/AAV9; or AAV5/AAV9 serotype.

An example of a method and means to deliver miRNA to target cells has been provided in WO2020/104469 A1, which is incorporated herein by reference.

In some embodiments, the concentration of the adeno-associated viral vector is at least 1E13 gc/mL; preferably at least 2E13 gc/mL; more preferably at least 1E14 gc/mL; for example at least 3E14 gc/mL. In some embodiments, the concentration of the adeno-associated viral vector is at least 6E14 gc/mL. In some embodiments, the concentration of the adeno-associated viral vector is between 4E14 gc/mL and 5E14 gc/mL.

In some embodiments, the formulation comprises an AAV5 with a transgene encoding a microRNA, and the concentration of AAV5 is at least 6E14 gc/mL.

In one example, the formulation comprises an AAV5 with a transgene encoding a microRNA, and the concentration of AAV5 is 3E14 gc/mL.

In another example, the formulation comprises an AAV5 with a transgene encoding a microRNA, and the concentration of AAV5 is 6E14 gc/mL.

In yet another example, the formulation comprises an AAV5 with a transgene encoding a microRNA, and the concentration of AAV5 is 7.5E14 gc/mL.

The stable, high concentration formulation may be particularly useful in delivering sufficient genome copies of the drug product when the dosage volume is limited. An example of such application is when injection into the cisterna magna (CM, whose average volume is approximately 1 ml only) is required.

In some embodiments, the formulation comprises an AAV9 with a transgene encoding a microRNA, and the concentration of AAV9 is at least 6E14 gc/mL.

In one example, the formulation comprises an AAV9 with a transgene encoding a microRNA, and the concentration of AAV9 is 3E14 gc/mL.

In another example, the formulation comprises an AAV9 with a transgene encoding a microRNA, and the concentration of AAV9 is 6E14 gc/mL.

In yet another example, the formulation comprises an AAV9 with a transgene encoding a microRNA, and the concentration of AAV9 is 7.5E14 gc/mL.

The formulation may also have a pH value compatible with human cerebrospinal fluid. For example, the formulation may have a pH of about 5 or higher, preferably about 7 or higher. In some embodiments, the formulation may have a pH value of from 5 to 8, preferably from 6 to 8, more preferably from 6.5 to 8, even more preferably from 7 to 8, for example from 7.1 to 7.7. In some embodiments of the present invention, the formulation has a pH value of 7.3.

Tonicity is a measure for the effective osmotic pressure that a liquid formulation can exert, and depends primarily on the number of dissolved particles in solution. Osmotic pressure is an important factor affecting biological cells. Hypertonicity is the presence of a solution that causes cells to shrink. Hypotonicity is the presence of a solution that causes cells to swell. Isotonicity is the presence of a solution that produces no change in cell volume. When a biological cell is in a hypotonic environment, the cell interior accumulates water, water flows across the cell membrane into the cell, causing it to expand. For mammalian cells this can lead to cytolysis, and tonicity is therefore important when fragile cells such as nerve cells or brain cells are to be exposed to a composition. Tonicity agents are therefore added to injectable preparations to prevent osmotic shock at the site of injection upon administration, and thereby reduce local irritation or even damage to the CNS. Typical tonicity agents are thus excipients used for tonicity adjustment, and are known in the art.

In some embodiments, the tonicity agent comprises a periodic group 1 or group 2 metal salt, preferably a periodic group 1 or group 2 metal chloride salt, preferably selected from the group consisting of NaCl, KCl, CaCl₂, MgCl₂, and combinations thereof. Preferably, the tonicity agent comprises NaCl. In one example of the invention, the tonicity agent comprises a combination of NaCl and KCl. Tonicity agents help to stabilize the formulation. Preferably, the tonicity agent is present in an amount of from 5 mM to 150 mM, preferably from 25 mM to 125 mM, more preferably from 75 mM to 125 mM, for example, 90 mM. For example, the tonicity agent may comprise at least 75 mM NaCl. In some embodiments, the tonicity agent is present in an amount of 90 mM. For example, the tonicity agent may comprise a combination of NaCl and KCl and be present in an amount of 90 mM. In some embodiments, the tonicity agent comprises at least 80 mM NaCl and at least 2 mM KCl. For example, the tonicity agent may comprise 3 mM KCl and 87 mM NaCl or 82 mM NaCl. Therefore, in some embodiments the tonicity agent is selected from the group consisting of NaCl, KCl, MgCl₂, CaCl₂, and combinations thereof; and is present in an amount of from 5 mM to 150 mM.

The tonicity agent is primarily used to establish a desired osmolality for the formulation. Preferably, the formulation is substantially isotonic to human cerebrospinal fluid. In particular, the formulation may have an osmolarity of from 250 to 330 mOsm/kg. In some preferred embodiments, the formulation has an osmolarity of from 270 to 320 mOsm/kg. For example, the formulation has an osmolarity of 280 mOsm/kg. Therefore, in one embodiment, the formulation has an osmolarity of from 250 to 330 mOsm/kg and a pH value of from 5 to 8.

Cryoprotectants are additives that help minimize polypeptide/biopharmaceutical denaturation when polypeptides are present in the composition. Cryoprotectants have been found to be particularly advantageous in providing a stable drug product that eliminates or substantially eliminates the formation of aggregates or agglomerates. In preferred embodiments, the cryoprotectant is a carbohydrate or a polymer. The term “carbohydrates” as used herein refers to both naturally occurring saccharides as well as to polyols that can be derived from saccharides. Examples of suitable carbohydrates are trehalose, sucrose, dextrose, dextran, and combinations thereof. Therefore, in some embodiments, the cryoprotectant is a carbohydrate selected from the group consisting of trehalose, sucrose, dextrose, dextran, and combinations thereof. Preferably, the cryoprotectant is trehalose or dextran. Trehalose is a disaccharide formed by a 1,1-glycosidic bond between two α-glucose units, and sucrose is a disaccharide that is O-α-D-glucopyranosyl-(1→2)-β-D-fructofuranoside. Optionally other disaccharides could be used in their place. Dextrose is D-glucose, and optionally other monosaccharides could be used in its place. In a more preferred embodiment of the invention, the cryoprotectant is trehalose. In another embodiment of the invention, the cryoprotectant is dextran, for example, dextran 6000. In some embodiments, the cryoprotectant is present in the formulation in an amount of up to 8% w/v, preferably from 2% w/v to 5% w/v, for example from 2% w/v to 3% w/v. In some embodiments, the cryoprotectant is trehalose and is present in the formulation in an amount of from 2% w/v to 6% w/v, for example, 3% w/v. In some embodiments, the cryoprotectant is dextran, for example, dextran 6000, and is present in an amount of from 1% w/v to 3% w/v, for example from 2% w/v to 2.5% w/v. Therefore, in some embodiments, the cryoprotectant is a carbohydrate selected from the group consisting of trehalose, sucrose, dextrose, dextran, and combinations thereof; and present in an amount of up to 8% w/v.

Surfactants and their characteristics are well known, and surfactants generally comprise at least one polar head group and at least one apolar or hydrophobic tail. The surfactants used here preferably comprise a single tail and preferably comprise a single head group. They are preferably charge neutral, which means that the surfactants do not have a net charge at the conditions for their use. Alkylated saccharides are most preferred for use in this invention, particularly polysorbates, more particularly polysorbate 20. In some embodiments, the surfactant is a non-ionic surfactant. Preferably, the surfactant is a polyethoxylated non-ionic surfactant such as a polysorbate. In a preferred embodiment of the invention, the surfactant is Polysorbate-20.

The surfactant has been found to be particularly advantageous in improving the stability and reducing the formation of aggregated or agglomerated particles in the formulation. Optionally, the surfactant may be present in an amount of from 0.001% v/v to 0.1% v/v, preferably from 0.03% v/v to 0.08% v/v, more preferably from 0.005% v/v to 0.02% v/v. In some embodiments, the surfactant is present in the formulation in an amount of from 0.005 to 0.015% v/v. Therefore, in some embodiments, the surfactant is a non-ionic surfactant and is present in an amount of from 0.0001% v/v to 0.1% v/v, preferably from 0.03% v/v to 0.08% v/v, more preferably from 0.005% v/v to 0.02% v/v. In some embodiments, the surfactant is present in the formulation in an amount of from 0.005 to 0.015% v/v, for example about 0.01% v/v. For example, the surfactant is polysorbate-20 and is present in an amount of about 0.01% v/v.

Buffering agents are known in the art, and help maintain the pH of the composition stable within a given range. A buffering agent is often a buffer salt. The buffer may be selected from the group consisting of an artificial cerebrospinal fluid (aCSF), phosphate buffer, acetate buffer, citrate buffer, HEPES buffer, Tris buffer, and combinations thereof.

In some embodiments, the buffer may be a phosphate buffer, for example, a phosphate buffer at a pH from 6.5 to 7.5. In some embodiments, the buffer may be an acetate buffer, for example, an acetate buffer at a pH from 5 to 5.5. In some embodiments, the buffer may be a citrate buffer, for example, a citrate buffer at a pH from 5.5 to 6.5. In some embodiments, the buffer may be a Tris buffer, for example, a Tris buffer at a pH from 7.5 to 8. In some embodiments, the buffer may be a HEPES buffer, for example, a HEPES buffer at a pH from 7 to 8. In some embodiments, the buffer may be aCSF for example, a aCSF at a pH from 7.0 to 7.6. For example, the aCSF may have the following final ion concentration (in mM): Na 150; K 3.0; Ca 1.4; Mg 0.8; P 1.0; C 155. Preferably, the buffer is a phosphate buffer or TRIS; for example, the buffer is a 10 mM phosphate buffer at pH 7.3.

The buffering agent, tonicity agent, cryoprotectant, and any other functionally defined components of the composition, such as a surfactant, can be individual substances, but can also be mixtures. For instance, the buffering agent can be a single phosphate salt such as Na₂HPO₄, but it can also be a mixture of substances such as a mixture of Na₂HPO₄ and KH₂PO₄. Similarly the tonicity agent can be a single substance such as NaCl, but it can also be a mixture such as a combination of NaCl and KCl. Throughout this document, when only a single substance is specified for a functional definition, preferably no other substances are present for that same function. The same holds for when a plurality is specified, in which case only the specified substances are comprised for that same function.

Some compounds, such as for example phosphate salts, can act as a buffering agent but can also act as a tonicity agent. As used herein, when a compound is present as one functionally defined component of the composition, then it preferably is not considered as also satisfying any requirements for further functionally defined components. Accordingly, when one or more of acetate salts, citrate salts, HEPES, TRIS, or phosphate salts are present, these are preferably considered as buffering agent only. When one or more of NaCl, KCl, MgCl₂, CaCl₂, or a mixture thereof are present, these are preferably considered as tonicity agent only. When one or more of trehalose, dextran, sucrose, dextrose, or poly(ethylene glycol) are present, these are preferably considered as cryoprotectant only. When one or more non-ionic surfactants are present, these are preferably considered as surfactants only.

In a particularly preferred embodiment, the formulation comprises, consists, or consists essentially of:
an adeno-associated viral vector with a transgene encoding a microRNA targeting ATXN3 mRNA;
about 5-30 mM Phosphate buffer at a pH from 7 to 8;
at least about 75 mM NaCl;
about 2-5 mM KCl;
about 2% w/v to 4% w/v trehalose;
about 0.005% v/v to 0.015% v/v PS-20.

For example, the formulation may comprise, consist or consist essentially of:
an adeno-associated viral vector with a transgene encoding a microRNA targeting ATXN3 mRNA;
about 9 mM Tris at a pH of about 7.3;
about 82 mM NaCl;
about 3 mM KCl;
about 3% w/v trehalose;
about 0.01% v/v PS-20 (Tween 20)

For example, the formulation may comprise, consist or consist essentially of:
an adeno-associated viral vector with a transgene encoding a microRNA targeting ATXN3 mRNA;
about 9 mM Tris at a pH of about 7.3;
about 87 mM NaCl;
about 3 mM KCl;
about 3% w/v trehalose;
about 0.01% v/v PS-20 (Tween 20)

In some embodiments, the formulation is substantially free of visible particles. Thus, the formulation is essentially free of particles visible to the naked human eye, as determined and characterized according to U.S. Pharmacopeial Convention (USP) Chapter 790 “VISIBLE PARTICULATES IN INJECTIONS” guidance of 2014 regarding parenteral medical products being “essentially free” of visible particulate matter (USP 790), which is incorporated herein by reference. Visible particles may be a sign of aggregation, agglomeration, and/or degradation of AAV particles, such that a lack of visible particles may be advantageous to indicate a particularly stable formulation free from substantial aggregation or agglomeration. Preferably, the formulation also comprises limited subvisible particles, as determined and characterized according to U.S. Pharmacopeial Convention (USP) Chapter 787 “SUBVISIBLE PARTICULATE MATTER IN THERAPEUTIC PROTEIN INJECTIONS” (USP 787), which is incorporated herein by reference. For example, the number of particles having a diameter of 25 μm is no more than 600 per vial, and the number of particles having a diameter of ≥10 μm is no more than 6000 per vial, in the formulation. Optionally, the formulation has a Dv90 of less than 50 μm, preferably less than 25 μm, more preferably less than 10 μm. The Dv90 values represent the percent (90% in this case) of the formulation that has a size no larger than the specified value.

In some embodiments, the formulation of the invention may be used in treating spinocerebellar ataxia (SCA), for example, spinocerebellar ataxia type 3 (SCA3). In some embodiments, the formulation is be administered by intra-CSF (cerebral spinal fluid) delivery. For example, the formulation is administered by intra-CSF delivery such as intracisternal delivery. Optionally, the formulation may be administered by direct injection to the brain, or by repeat injection at the cisterna magna, or by slow infusion at the cisterna magna, or by a combination of direct injection to the brain and injection(s) at the cisterna magna. In some embodiments, the formulation may be administered by injection at the cisterna magna. In some embodiments, the formulation may be administered by a single injection at the cisterna magna. For example, the formulation may be administered by (a single) injection (optionally CT-guided) into the cisterna magna over 5 minutes.

The “cisterna magna” or “cerebellomedullary cistern” is one of three principal openings in the subarachnoid space between the arachnoid and pia mater layers of the meninges surrounding the brain. The openings are collectively referred to as cisterns. The cisterna magna is located between the cerebellum and the dorsal surface of the medulla oblongata. Cerebrospinal fluid produced in the fourth ventricle drains into the cisterna magna via the lateral apertures and median aperture.

In some embodiments, the formulation is administered at 5 mL per subject at a concentration of the adeno-associated viral vector of 3E14 gc/mL. In some specific embodiments, the formulation is administered at 5 mL per injection site at a concentration of the adeno-associated viral vector of 3E14 gc/mL. In some embodiments, the formulation is administered at a dosage of from 4E14 to 5E15 genome copies, preferably from 8E14 to 2E15 genome copies, more preferably from 1E15 to 2E15 genome copies, per subject. For example, the formulation is administrated at a dosage of 1.5E15 genome copies per subject.

In some embodiments, the formulation of the invention may be administered together with a contrast agent visualisable by magnetic resonance imaging (MRI). This may be advantageous when it is desired to monitor the patient's brain by MRI.

Alternatively, or in addition as another product of interest, the nucleotide sequence comprising the transgene encoding a microRNA as defined herein above may further comprise a nucleotide sequence encoding a polypeptide that serves as a selection marker protein to assess cell transformation and expression. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel, see below.

Furthermore, the nucleotide sequence comprising the transgene as defined herein above may comprise a further nucleotide sequence encoding a polypeptide that may serve as a fail-safe mechanism that allows to cure a subject from cells transduced with an AAV vector of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the transgenic cells in the subject (see e.g. Clair et al., 1987, Antimicrob. Agents Chemother. 31: 844-849).

In one aspect the invention provides a method for preparing a drug product for administration to the central nervous system, the method comprising the steps of:
i) providing a formulation according to the invention; and
ii) aliquoting the formulation into a suitable dosage form.

The prepared drug product is suitable for administration to the central nervous system, for instance as described elsewhere herein. Accordingly, the method is suitable for preparing formulations for use according to the invention, preferably wherein the composition is for intra-cerebrospinal fluid administration. Therefore, in one embodiment the formulation for use according to the invention is administered to a patient's cisterna magna.

In yet another aspect of the invention, the present invention provides a method of treating SCA, in particular SCA3, by administering the formulation of the invention to a patient's brain.

### Definitions

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a combination or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements.

The indefinite article “a” or “an” thus usually means “at least one”.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The word “about” or “approximately” when used in association with a numerical value (e.g. about 10) preferably means that the value may be the given value (of 10) more or less 10% of the value, optionally more or less 5%.

Each embodiment as identified herein may be combined together unless otherwise indicated. The invention has been described above with reference to a number of embodiments. A skilled person could envision trivial variations for some elements of the embodiments. These are included in the scope of protection as defined in the appended claims. All patent and literature references cited are hereby incorporated by reference in their entirety.

In addition, the following terms are used herein and defined as follows.
“Capsid”—A protein shell surrounding viral DNA, RNA, or microRNA that helps target the genetic material to specific cell types.
“Gene cassette”—A small fragment of DNA, RNA, or microRNA containing the therapeutic gene and instructions for the cell on how to use the gene.
“Vector”—This may refer to the DNA, RNA, or microRNA molecule itself or the carrier construct containing the DNA, RNA, or microRNA to be delivered to a cell.
“microRNA” (also termed “miRNA” or “siRNA”)—a small single-stranded non-coding RNA molecule, typically containing a guide strand as described above.
“SCA”—spinocerebellar ataxia.
“substantially”—the term “substantially” as used herein is a broad term, and is to be given its ordinary and customary meaning to a person of ordinary skill in the art (and is not to be limited to a special or customized meaning), and refers without limitation to being largely but not necessarily wholly that which is specified.
“PS-20”—polysorbate-20.
“iPSCs”—induced pluripotent stem cells.
“ITR”—inverted terminal repeat.
“MRI”—magnetic resonance imaging.

Specific embodiments of the present invention are described in the following paragraphs.
1. An isotonic pharmaceutical formulation comprising:
   a buffer
   an adeno-associated viral vector with a transgene encoding a microRNA;
   a tonicity agent;
   a cryoprotectant; and
   a surfactant.
2. The formulation of paragraph 1, wherein the formulation has an osmolarity of from 250 to 330 mOsm/kg, preferably from 260 to 310 mOsm/kg, for example from 270 to 300 mOsm/kg.
3. The formulation of paragraph 1 or 2, wherein formulation has a pH value of from 5 to 8, preferably from 6 to 8, more preferably from 6.5 to 8, for example from 7 to 8.
4. The formulation of any one of the preceding paragraphs, wherein the formulation is substantially free of visible particles.
5. The formulation of any one of the preceding paragraphs, wherein the adeno-associated viral vector with a transgene encoding microRNA targets ATXN mRNA, for example, ATXN3 mRNA.
6. The formulation of any one of the preceding paragraphs, wherein the adeno-associated viral vector comprises an AAV2 serotype, an AAV5 serotype, an AAV9 serotype, a hybrid AAV serotype, or a combination thereof, preferably the adeno-associated viral vector comprises an AAV5 serotype or an AAV9 serotype, for example, an AAV5 serotype.
7. The formulation of any one of the preceding paragraphs, wherein the concentration of the adeno-associated viral vector is at least 1E13 gc/ml, such as at least 3E13 gc/ml. In some embodiments, the concentration of the adeno-associated viral vector is between 0.5E13 and 6E13 gc/ml, such as between 1E13 and 5E13 gc/ml.
8. The formulation of any one of the preceding paragraphs, wherein the cryoprotectant is a sugar.
9. The formulation of paragraph 8, where the cryoprotectant is a sugar selected from the group consisting of trehalose, sucrose, dextrose, dextran, and combinations thereof; for example, the cryoprotectant is trehalose; alternatively, the cryoprotectant is a dextran, such as a dextran 6000.
10. The formulation of any one of the preceding paragraphs, wherein the cryoprotectant is present in an amount of up to 8% w/v preferably from 2% w/v to 5% w/v, for example from 2% w/v to 3% w/v.
11. The formulation of any one of the preceding paragraphs, wherein the cryoprotectant is trehalose and is present in an amount of 3% w/v.
12. The formulation of any one of the preceding paragraphs, wherein the tonicity agent is selected from the group consisting of NaCl, KCl, MgCl₂, CaCl₂, and combinations thereof.
13. The formulation of paragraph 12, wherein the tonicity agent is a combination of NaCl and KCl,
14. The formulation of any one of the preceding paragraphs, wherein the tonicity agent is present in an amount of from 5 mM to 150 mM, preferably from 25 mM to 125 mM, more preferably from 75 mM to 125 mM, for example, 90 mM.
15. The formulation of paragraph 14, wherein the tonicity agent comprises at least 75 mM NaCl.
16. The formulation of any one of the preceding paragraphs, wherein the surfactant is a non-ionic surfactant, preferably a polyethoxylated non-ionic surfactant such as a polysorbate, for example, polysorbate-20.
17. The formulation of any one of the preceding paragraphs, wherein the surfactant is present in an amount of from 0.001% v/v to 0.1% v/v, preferably from 0.003% v/v to 0.08% v/v, more preferably from 0.005% v/v to 0.02% v/v, for example about 0.01% v/v.
18. The formulation of any one of the preceding paragraphs, wherein the surfactant is polysorbate 20 and is present in an amount of 0.01% v/v.
19. The formulation of any one of the preceding paragraphs, wherein the buffer is selected from the group consisting of an artificial cerebrospinal fluid (aCSF), acetate buffer, citrate buffer, phosphate buffer, HEPES, Tris, and combinations thereof; preferably, the buffer is a phosphate buffer or TRIS; for example, the buffer is a 9-10 mM phosphate buffer at pH 7.3.
20. The formulation of any one of the preceding paragraphs, wherein the formulation remains stable at room temperature without substantial formation of aggregates or agglomerates for at least 12 hours, preferably at least 18 hours, for example at least 24 hours, after thawing.
21. The formulation of any one of the preceding paragraphs for use in treating Spinocerebellar Ataxia (SCA), for example, SCA type 1, SCA type 2, or SCA type 3, preferably SCA type 3.
22. The formulation for use of paragraph 21, wherein the formulation is administered by intra-CSF (cerebral spinal fluid) delivery; preferably, the formulation is administered by intracisternal delivery.
23. The formulation for use of paragraph 21 or 22, wherein the formulation is administered to a patient's cisterna magna.
24. The formulation for use of any one of paragraphs 21 to 23, wherein the formulation is administered together with contrast agent for MRI monitoring of the administration.
25. A method of treating Spinocerebellar Ataxia by administering the formulation of any one of the preceding paragraphs to a patient.
26. The method of paragraph 25, wherein the formulation is administered by a single dosage delivered by intracisternal injection into the patient.
27. The method of paragraph 25 or 26, wherein the formulation is administered together with a contrast agent visualisable by MRI.

### FIGURES

Specific embodiments of the present invention will be described by way of example, with reference to the accompanying drawings in which:

FIG. 1 shows results of Example 1. Tm (melting temperature of the capsid as determined by iDSF approach) as a function of the accelerated stress conditions and duration.

FIG. 2 shows protein content and fluorescence as a function of the stress conditions and durations for the different salt concentrations in Example 1. The NaCl concentrations are in different greyscale shades. The different buffers are pooled. UV280 content and Trp_Fluo:280,335 are in arbitrary units. For the storage at 25° C. and 40° C. the scale is in days, 7 and 2 respectively, while for the shaking is for 24 hours.

FIG. 3 is a Production profiler from a DoE experiment based on the absorbance and fluorescence response on the full dataset of all timepoints and all stresses.

FIG. 4 is a Production profiler from a DoE experiment based on the absorbance and fluorescence response on the full dataset of all timepoints and all stresses.

FIG. 5 shows poly-dispersity index (PDI) measurements by dynamic light scattering as a function of fold concentration.

FIG. 6 shows z-average measurements by dynamic light scattering as a function of fold concentration.

FIG. 7 shows z-average measurements by dynamic light scattering of accelerated- and long-term stability.

FIG. 8 A/B show a subvisible particle analysis of accelerated- and long-term stability.

FIG. 9 is a visual inspection overview of AAV9 DP in five formulation buffers at different stress conditions. No fill: no visible particulates present; Diagonal line fill: one-two visible particulates present; Dense diagonal line fill: few visible particulates present.

FIG. 10 shows luciferase activity in cell lysates of Huh-7 cells transduced with AAV9 (1489)-luciferase formulated in different buffers.

### EXAMPLES

### Abbreviations

AAV is Adeno Associated Virus
AC is Affinity Chromatography
aCSF is Artificial Cerebrospinal Fluid
AIM is Analytical Investigational Method
CSF is Cerebrospinal Fluid
DLS is Dynamic Light Scattering
DoE is Design of Experiments
DP is Drug Product
DPD is Drug Product Development
DS is Drug Substance
F/T is Freeze/Thaw
Gc is Genome Copies
GMP is Good Manufacturing Practice
iDSF is Intrinsic Differential Scanning Fluorimetry
IEX is Ion Exchange
MW is Molecular weight
N/A is Not Applicable
NF is Nanofiltration
PD is Process Development
PDI is Polydispersity index
PEG is Poly-ethylene Glycol
R.U. is Arbitrary units
SOP is Standard Operational Procedure
STR is Stirring Tank Reactor
Tp is Total Particles
UF/DF is Ultrafiltration/Diafiltration
VI is Visual Inspection

### Materials and Methods

The following materials were used in the examples and are available as specified below in Table 1.

TABLE 1
Materials used in the examples and their suppliers

| Raw Materials | Supplier |
|---|---|
| Acetic Acid | Merck glacial 100%, |
| Citric acid | Merck |
| Dextran 20000 | Sigma |
| Dextran 55000 | Sigma |
| Dextran 6000 | Sigma |
| Dextran 70000 | Sigma |
| Dextrose | Sigma |
| Ethanol 96% | Fisher Chemical |
| Formulation Buffer PS-20 | J. T. Baker |
| HCl (37%) | Merck |
| Hepes | Sigma |
| Hepes Sodium Salt | Sigma |
| K₂HPO₄ (salt) | MP Biomedicals |
| KCl | Merck |
| KH₂PO₄ (acid) | Merck |
| L-Histidine | Sigma |
| MgCl₂—6H₂O | Sigma |
| NaCl | Merck |
| PEG 200 | Sigma |
| PEG 20000 | Sigma |
| PEG 35000 | Sigma |
| PEG 4000 | Sigma |
| Polysorbate 20 (PS-20) | J. T. Baker |
| Sodium Acetate | Merck |
| Sodium citrate tribasic dihydrate | Sigma-Aldrich |
| Sodium phosphate dibasic | Merck |
| Sucrose | Merck |
| Trehalose | Sigma |
| Tris | Merck |

### Measurements

The read-outs obtained were as follows:
Absorbance at 260 nm, which correlates with DNA concentration.
Absorbance at 280 nm, which correlates with protein concentration.
Absorbance at 350 nm (turbidity), which correlates with protein aggregation and/or particle formation.
Absorbance at 900 nm, which correlates with the background signal from the plastic of the wells and seal.
Absorbance at 975 nm measures the water absorbance, thus, the path length.
Tryptophan fluorescence at 280/335 nm, measured from the top and the bottom of the micro-well plate. This measurement correlates with protein and/or capsid unfolding and denaturation. The top read-out was used in the figures.

The protein and DNA concentrations were obtained as follows:

According to the Lambert-Beer law, the absorbance is proportional to the concentration and the light path length. A constant named the extinction coefficient turns this into an equation.

A=cc·L·ε, where A is the absorbance, cc is the concentration, L is the light path length and c is the extinction coefficient.

The light pathlength is measured by the water absorbance measured at 975 nm (A975) minus the plastic background measured at 900 nm (A900) thus:

L=A975−A900

The protein concentration is measured by measuring the absorbance at 280 nm (A280), thus:

A280=cc(protein)·L·ε(protein)

The background from the precipitated and aggregated proteins or turbidity is measured by the absorbance at 350 nm (A350). It is assumed to be similar to the contribution of turbidity at 280 nm, and thus, deducted from the absorbance at 280 nm. Then:

$$cc ( protein ) . ε ( protein ) = ( A 280 - A 350 ) / ( A 975 - A 900 )$$

Since ε (protein) is a constant, we can conclude that cc (protein)·ε (protein), is proportional to the protein concentration, which is computed from the different absorbance values measured (A280, A350, A975 & A900). For the DNA concentration at 260 nm, similar reasoning was applied.

Intrinsic Differential Scanning Fluorimetry (iDSF) Measurements Using Nanotemper® Technology

The denaturation temperature of the capsids was followed using the Nanotemper® technology by which a sample was heated at increasing temperatures and the fluorescence of the capsids followed. As the capsids denature and expose fluorescent amino acids to the solvent environment, the fluorescence diminishes as the environment changes from a rigid one, inside the protein, to a less rigid one. The ratio between the fluorescence at 335 nm and 350 nm is computed to make the measurements independent of the concentrations.

### Example 1—Formulation Study AAV5

The drug product (DP) used in this example was AAV5 with a transgene encoding a microRNA targeting AAXN3 mRNA at a concentration of 4.7E13 gc/mL.

rAAV5-miAAXN3s was buffer exchanged in a multi-well system into thirty different formulations comprising different pHs, buffer types, and salt contents using 96 well filter plates (100 kDa cut-off). After the buffer was exchanged and filtered through a 0.2 μm filter, the formulations were aliquoted into four plates according to the plate layout and buffer formulation compositions. The final concentration of the AAV5 in the wells was 1.0E13 gc/mL. Each formulation is prepared in duplicate. The outer rows and columns are filled with WFI to rule out plate location effects. The buffers have overlapping pHs, to differentiate between a buffer and a pH effect.

Each plate was assigned to a specific stress condition: F/T at 80° C., storage at 25° C. and 65° C. and shaking at RT. The accelerated studies comprised the following:
Freeze at −80° C. (at least 1 h) and thaw at RT (1 hour) for 1, 3 & 10 cycles. The table with the experiments dates show when each F/T cycle was carried out.
Shaking at RT for 1 and 6 hours. There are two shaking measurements in the experiments due to a deviation. The first shake experiment was not spun down before the measurements, thus, a second shaking plate was added which underwent the spinning down and the shaking at RT for 1 and 6 hours.
Storage at 25° C. for 2, 7, and 14 days.
Storage at 65° C. for 2, 7, and 14 days.
For all the above-mentioned conditions a micro-well plate was prepared and initial measurements (T=0) were carried out.

The T=0 protein contents as followed by Absorbance at 280 nm (corrected for turbidity and path-length in four multi-well plates; arbitrary units) are shown in Table 2. The T=0 protein contents as followed by Fluorescence at 335 nm (top measurement from four multi-well plates; arbitrary units) are shown in Table 3.

TABLE 2
The T = 0 protein contents as followed by Absorbance at 280 nm (A: F/T
at −80° C.; B: Shaking; C: 25° C.; D: 65° C.)

| | | Buffer | | | | | | | | | |
|---|---|---|---|---|---|---|---|---|---|---|---|
| | | Acetate | | | Citrate | | | Phosphate | | | Tris |
| | pH | 5 | 5.5 | 5.5 | 6 | 6.5 | 6.5 | 7 | 7.5 | 7.5 | 8 |
| A. | | | | | | | | | | | |
| | WFI | 0.8844 | 0.9551 | 1.7323 | 1.6212 | 1.7374 | 1.6010 | 1.7922 | 1.7200 | 1.5835 | 1.6015 |
| Salt | 0.075M NaCl | 1.4720 | 1.5909 | 1.5459 | 1.6280 | 1.6263 | 1.4016 | 1.6465 | 1.6179 | 1.4414 | 1.7020 |
| | 0.15M NaCl | 1.5859 | 1.6733 | 1.5100 | 1.6299 | 1.6987 | 1.4634 | 1.7135 | 1.6517 | 1.4899 | 1.5455 |
| B. | | | | | | | | | | | |
| | WFI | 0.7788 | 0.8592 | 1.4826 | 1.4898 | 1.5025 | 1.3705 | 1.3990 | 1.5253 | 1.4321 | 1.5303 |
| Salt | 0.075M NaCl | 1.4309 | 1.5458 | 1.5209 | 1.4575 | 1.5794 | 1.4562 | 1.4661 | 1.5478 | 1.5581 | 1.5228 |
| | 0.15M NaCl | 1.4367 | 1.6224 | 1.6181 | 1.4415 | 1.6447 | 1.6499 | 1.5224 | 1.6433 | 1.5680 | 1.4873 |
| C. | | | | | | | | | | | |
| | WFI | 0.7636 | 0.8480 | 1.5426 | 1.5396 | 1.5550 | 1.4405 | 1.4446 | 1.5819 | 1.5203 | 1.5673 |
| Salt | 0.075M NaCl | 1.4487 | 1.5833 | 1.6106 | 1.4319 | 1.6252 | 1.4550 | 1.4802 | 1.5818 | 1.5716 | 1.5745 |
| | 0.15M NaCl | 1.5204 | 1.752 | 1.8037 | 1.5347 | 1.7079 | 1.7423 | 1.6641 | 1.6965 | 1.6468 | 1.5618 |
| D. | | | | | | | | | | | |
| | WFI | 0.7850 | 0.8692 | 1.5101 | 1.5354 | 1.5585 | 1.4719 | 1.4643 | 1.6042 | 1.5561 | 1.7406 |
| Salt | 0.075M NaCl | 1.4500 | 1.5929 | 1.5784 | 1.4394 | 1.6313 | 1.4770 | 1.5101 | 1.6010 | 1.5878 | 1.6010 |
| | 0.15M NaCl | 1.4968 | 1.7490 | 1.7500 | 1.5500 | 1.7437 | 1.7489 | 1.6600 | 1.7079 | 1.6734 | 1.5718 |

TABLE 3
The T = 0 protein contents as followed by Fluorescence at 335 nm (A: F/T
at −80° C.; B: Shaking; C: 25° C.; D: 65° C.)

| | | Buffer | | | | | | | | | |
|---|---|---|---|---|---|---|---|---|---|---|---|
| | | Acetate | | | Citrate | | | Phosphate | | | Tris |
| | pH | 5 | 5.5 | 5.5 | 6 | 6.5 | 6.5 | 7 | 7.5 | 7.5 | 8 |
| A. | | | | | | | | | | | |
| | WFI | 1001 | 946 | 5451 | 5347 | 5503 | 4359 | 5439 | 5652 | 4826 | 6079 |
| Salt | 0.075M NaCl | 4682 | 5341 | 5153 | 5938 | 6029 | 4715 | 6160 | 6010 | 4404 | 6001 |
| | 0.15M NaCl | 5227 | 5806 | 4982 | 5879 | 6443 | 4988 | 6395 | 6254 | 5240 | 5625 |
| B. | | | | | | | | | | | |
| | WFI | 816 | 1204 | 4727 | 4846 | 5105 | 4302 | 4578 | 5302 | 4666 | 5296 |
| Salt | 0.075M NaCl | 4132 | 4882 | 4957 | 4500 | 5516 | 4811 | 4976 | 5542 | 5588 | 5417 |
| | 0.15M NaCl | 4212 | 5261 | 5437 | 4621 | 5887 | 5815 | 5171 | 6012 | 5536 | 5334 |
| C. | | | | | | | | | | | |
| | WFI | 859 | 1264 | 4906 | 5262 | 5560 | 4789 | 4886 | 5744 | 5271 | 5540 |
| Salt | 0.075M NaCl | 4404 | 5204 | 5113 | 4688 | 5939 | 4955 | 5288 | 5903 | 5829 | 5918 |
| | 0.15M NaCl | 4577 | 5870 | 6015 | 5165 | 6442 | 6305 | 5918 | 6436 | 6160 | 5685 |
| D. | | | | | | | | | | | |
| | WFI | 849 | 1234 | 4917 | 5173 | 5495 | 4651 | 4738 | 5616 | 5161 | 5470 |
| Salt | 0.075M NaCl | 4407 | 5154 | 5150 | 4588 | 5800 | 4882 | 5210 | 5788 | 5759 | 5816 |
| | 0.15M NaCl | 4548 | 5839 | 6001 | 5176 | 6364 | 6270 | 5865 | 6359 | 6009 | 5633 |

Table 2 clearly shows that the four multi-well plates have similar values, although not identical, for the corrected absorbance at 280 nm (protein concentration) as expected. In addition, for the four plates at T=0, the absorbances are higher in the presence of 150 mM NaCl for almost all of the conditions, therefore showing a higher protein content and a lower degree of aggregation. This can be concluded since the values measured include the correction for the background. On the whole, it is observed that the effects of the different formulations take place immediately at T=0 during the buffer exchange.

In Table 3, it is observed that there is a stabilizing salt effect at both 75 mM and 150 mM NaCl for most of the conditions. Acetate at pH 5 and 5.5 shows mostly destabilizing effects, especially without NaCl. Phosphate at pH 7.5 and citrate at pH 6.5 seem to favour the folded conformations. As above, the fact that the effects are observed at T=0 suggests that these take place during the buffer exchange step.

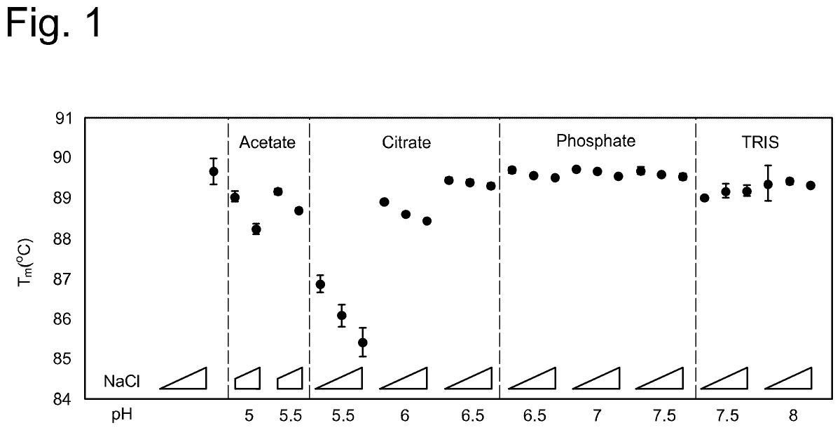

Results of the accelerated studies are shown in FIG. 1. These results point out the importance of the pH in the formulations as well as the effects of salt concentrations.

The overall observations are:
The effect of all tested buffers (at 20 mM: acetate, citrate, phosphate, and Tris), on the stability of the drug product measured by analytical assays: absorbance, and fluorescence, is seen immediately at T=0.
The lack of salt and buffer is detrimental to the stability of the drug product.
Salt (NaCl) addition, especially at a concentration level 75 mM or higher is beneficial for a formulation stabilizing effect.
A neutral or slightly basic formulation pH (7.0-7.5) is better for the stability of the drug product.
Formulations with basic buffer capacity, such as Tris, are more stabilizing when compared with buffered formulations with lower pHs.

Formulations with NaCl in buffers at a neutral or slightly basic pH (7.5) are used for further tests and optimization.

### Excipient Screening Study

According to the result of above, neutral to slightly basic pHs and the inclusion of salt support the stability of the capsids. Three buffers were chosen: phosphate, Tris, and Hepes, to support a pH equal to 7.5. As a control, artificial cerebrospinal fluid (aCSF) was used. 117 formulations in different buffers, with increasing concentrations of NaCl and defined concentrations of the putative excipients, were prepared; the latter were high enough to impact the viscosity but low enough not to have a large impact on the osmolality. The different excipients tested and their concentrations are shown in Table 4 below, among them surfactants such as various PEGs, and cryoprotectants such as trehalose, sucrose, dextrose, and dextrans. The polymer excipients were chosen to increase the viscosity of the formulations.

TABLE 4
Excipients and their corresponding concentrations

| Excipient | Example 2 (1) % w/v | Example 2 (2) % w/v |
|---|---|---|
| Sucrose | 4 | |
| Trehalose | 4 | 4 and 8 |
| Dextrose | 4 | 2 and 4 |
| Dextran 6000 | 3 | 1.5 and 3 |
| Dextran 20000 | 3 | |
| Dextran 70000 | 3 | |
| Dextran 550000 | 3 | |
| PEG 200 | 3 | |
| PEG 4000 | 3 | |
| PEG 20000 | 3 | |
| PEG 35000 | 3 | |

In the first excipient screening four sets of multi-well plates were prepared.

For the second excipients screening 18 formulations were prepared in glass vial containers. These comprise three buffers, as before, Tris, HEPES, and phosphate, and three putative excipients: trehalose, Low MW Dextran, and dextrose.

The plates of the first excipient screening and the vials of the second excipient screening were subject to accelerated studies at different stresses: F/T, shaking, and storage at two different temperatures (25° C. and 40° C.) which comprised the following durations/cycles as specified in Table 5 below:

TABLE 5
Accelerated stresses test conditions used

| | First Screening | Second Screening |
|---|---|---|
| Stress | Experiment | Experiment |
| Storage at 25° C. | 2 and 7 days | 7 days |
| Storage at 40° C. | 2 and 7 days | 2 days |
| Freeze at −80° C. (at least 1 h) | 1, 3 & 10 cycles | 4 cycles |
| and Thaw at RT (1 hour) | | |
| Shaking | 1 and 6 hours on | 24 hours at |
| | the vortex at | 200 rpm shaker |
| | maximum speed | |

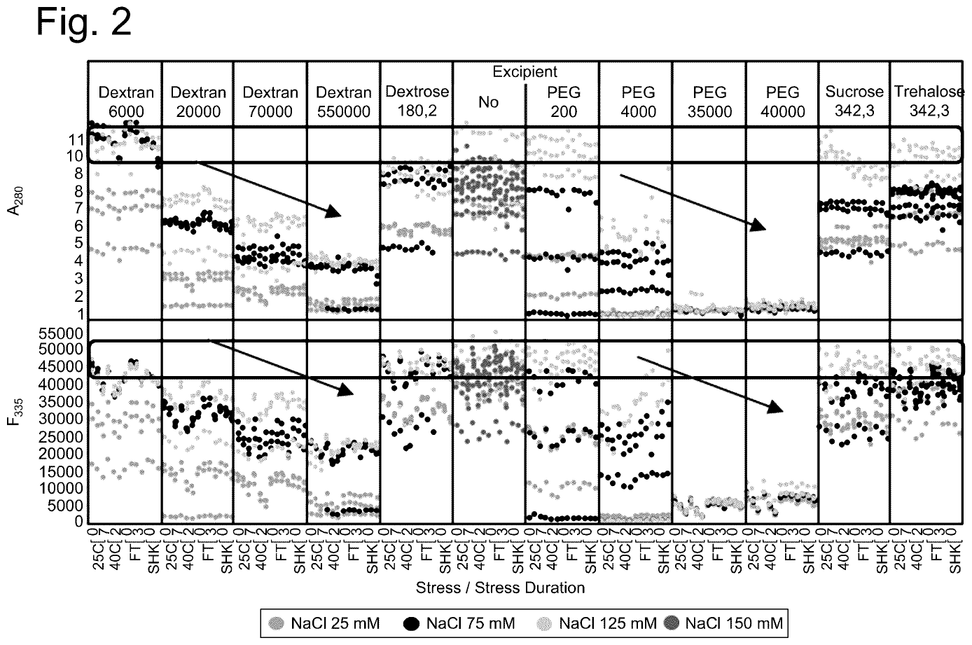

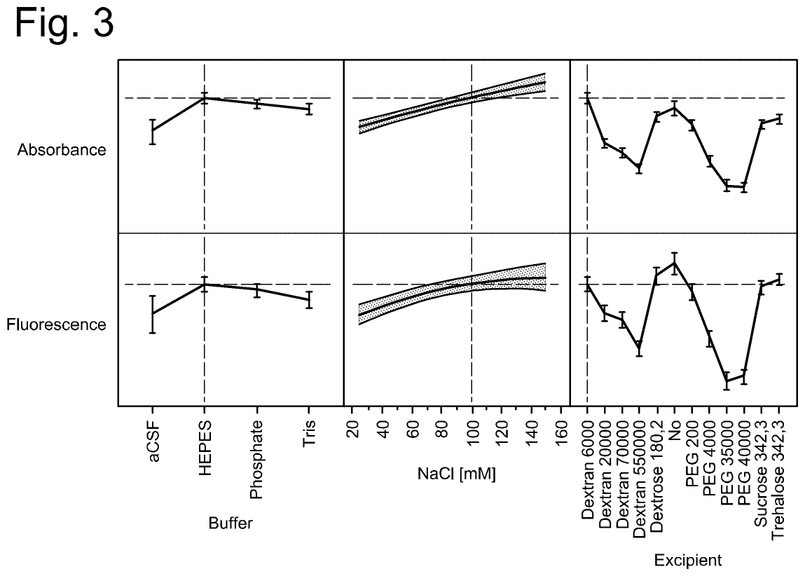

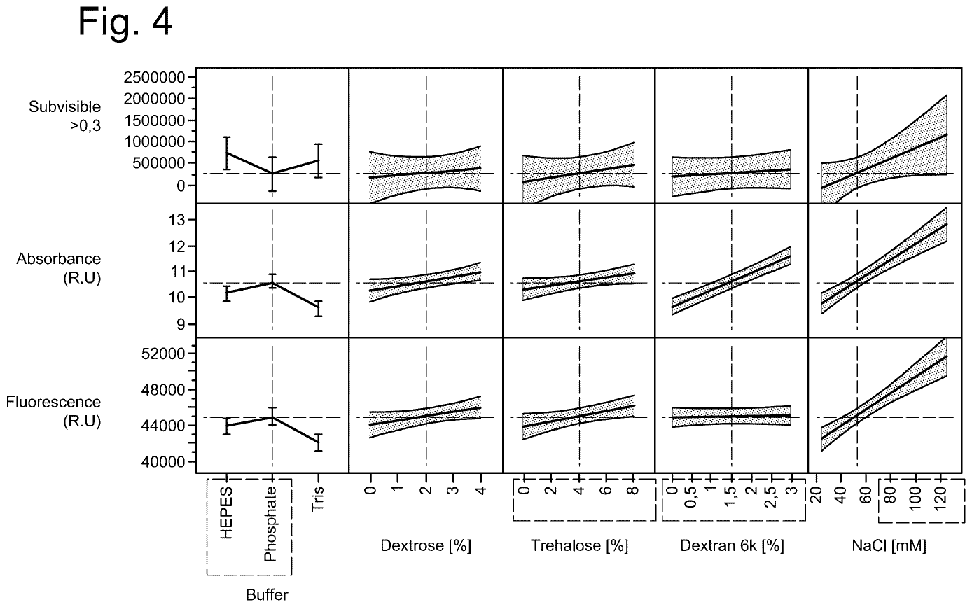

The protein content and fluorescence data as a function of the stress conditions and duration are shown in FIG. 2. A DoE experimental design was also carried out with the JMP® software and the prediction profilers are shown in FIGS. 3 and 4. The results suggest that higher salts have a positive effect while the effect of the buffers show that the most favourable buffers are phosphate and HEPES.

The overall observations are:
The buffer effects are immediate, take place at the buffer exchange step, and are observed at T=0;
High salt concentrations are beneficial for the stability of the samples, as well as slightly alkaline buffers;
Dextran 6000 and trehalose have a better stabilizing effect than dextrose
HEPES and phosphate are slightly better than Tris.

The following buffer and excipient choices are used for further tests and optimizations.
HEPES and phosphate as buffers
Dextran 6000 and trehalose as sugar excipients.

### Point of Aggregation Study

The four buffers used in the formulations are as specified in Table 6 below;

TABLE 6
Buffer compositions used

| | |
|---|---|
| Diafiltration Buffer A | HEPES 10 mM pH 7.3 |
| | 80 mM NaCl |
| | 3 mM KCl |
| | 1.4 mM CaCl₂ |
| | 0.8 mM MgCl₂ |
| | 3% w/v Trehalose |
| Diafiltration Buffer B | HEPES 10 mM pH 7.3 |
| | 123 mM NaCl |
| | 3 mM KCl |
| | 1.4 mM CaCl₂ |
| | 0.8 mM MgCl₂ |
| | 2.2% w/v Dextran 6000 |
| Diafiltration Buffer C | Phosphate 10 mM pH 7.3 |
| | 82 mM NaCl |
| | 3 mM KCl |
| | 0.8 mM MgCl₂ |
| | 3% w/v Trehalose |
| Diafiltration Buffer D | Phosphate 10 mM pH 7.3 |
| | 124 mM NaCl |
| | 3 mM KCl |
| | 0.8 mM MgCl₂ |
| | 2.2% w/v Dextran 6000 |

These formulations were concentrated stepwise by diafiltration. Samples were collected after each concentration step. Table 7 below shows the concentration folds and expected concentration.

TABLE 7
Sequential concentration steps applied to the different formulations

| Fold Concentration | Volume of Retentate | Expected concentration |
|---|---|---|
| during PoA | (mL) | (Gc/mL) |
| DF retentate | 300 | 5E13 |
| (Start sample) | | |
| 1, 5 | 200 | 7.5E13 |
| 2 | 150 | 1E14 |
| 4 | 75 | 2E14 |
| 6 | 50 | 3E14 |
| 12 | 25 | 6E14 |
| 15 | 20 | 7.5E14 |
| 20 | 15 | 1E15 |

The measurements followed after each concentration step were the following:
Genome copies per mL (Gc/mL)
Total particles per mL (TP/mL)
Visual inspection at T=0 and after 24 hours at 4° C. and two freeze and thaw (F/T) cycles.
Sub-visible particles by AccuSizer®.
Poly-dispersity index by Dynamic light scattering.

Table 8 below shows the visible inspection as a function of the fold concentration for the four formulations also after stresses, such as storage for 24 hrs at 4° C. or after two F/T cycles. It is observed that up to a six-fold concentration the phosphate-containing formulations perform better than the HEPES-containing formulations. It is also observed that the trehalose-containing formulations perform better than the Dextran 6000-containing formulations after a six-fold concentration step.

TABLE 8
Visible particle inspection as a function of the fold concentration after 4° C. storage and F/T cycles.

| | HEPES/Trehalose | | | HEPES/Dextran | | | Phosphate/Trehalose | | | Phosphate/Dextran | | |
|---|---|---|---|---|---|---|---|---|---|---|---|---|
| Fold | | 24 H | | | 24 H | | | 24 H | | | 24 H | |
| concentration | T₀ | 4° C. | F/T | T₀ | 4° C. | F/T | T₀ | 4° C. | F/T | T₀ | 4° C. | F/T |
| 0 | clear | clear | clear | clear | clear | clear | clear | clear | clear | clear | clear | clear |
| 1.5 | clear | clear | clear | clear | clear | clear | clear | clear | clear | clear | clear | clear |
| 2 | clear | clear | clear | clear | clear | clear | clear | clear | clear | clear | clear | clear |
| 4 | clear | clear | clear | clear | clear | clear | clear | clear | clear | clear | clear | clear |
| 6 | clear | clear | clear | clear | clear | collapsed | clear | clear | clear | clear | clear | clear |
| 12 | clear | turbid | clear | clear | collapsed | collapsed | clear | clear | clear | clear | clear | collapsed |
| 15 | | | | | | | clear | clear | clear | clear | collapsed | collapsed |

In Table 9 below, it is observed that the number of particles increases as the different formulations are concentrated. Furthermore, all the formulations show very similar results until the six-fold concentration step, which defines the largest concentration possible as described above. Only on the phosphate-containing formulations concentration steps up to 15-fold were carried out, which show an increment in the number of particles as expected.

TABLE 9
Sub-visible particles as a function of the fold concentration

| | Fold | Subvisible particles | | | | | |
|---|---|---|---|---|---|---|---|
| Formulation | concentration | >0.497 μm | >1.013 μm | >2.067 μm | >5.036 μm | >10.27 μm | >25.027 μm |
| HEPES/ | 0 | 2585 | 299 | 57 | 5 | 0 | 0 |
| Trehalose | 1.5 | 4945 | 110 | 11 | 0 | 0 | 0 |
| | 2 | 2753 | 111 | 38 | 0 | 0 | 0 |
| | 4 | 92897 | 1053 | 223 | 79 | 27 | 0 |
| | 6 | 45315 | 955 | 95 | 5 | 0 | 0 |
| | 12 | 53918 | 1210 | 127 | 22 | 0 | 0 |
| HEPES/ | 0 | 2279 | 185 | 15 | 0 | 0 | 0 |
| Dextran | 1.5 | 863 | 98 | 27 | 0 | 0 | 0 |
| | 2 | 7607 | 157 | 17 | 0 | 0 | 0 |
| | 4 | 19156 | 432 | 34 | 0 | 0 | 0 |
| | 6 | 118089 | 1849 | 99 | 13 | 0 | 0 |
| | 12 | 44445 | 1724 | 169 | 38 | 4 | 0 |
| Phosphate/ | 0 | 2022 | 126 | 36 | 0 | 0 | 0 |
| Trehalose | 1.5 | 2054 | 395 | 26 | 0 | 0 | 0 |
| | 2 | 2001 | 256 | 17 | 0 | 0 | 0 |
| | 4 | 11288 | 498 | 117 | 22 | 0 | 0 |
| | 6 | 16078 | 523 | 73 | 21 | 0 | 0 |
| | 12 | 21411 | 667 | 80 | 0 | 0 | 0 |
| | 15 | 32578 | 1873 | 371 | 82 | 29 | 8 |
| Phosphate/ | 0 | 3261 | 278 | 79 | 12 | 0 | 0 |
| Dextran | 1.5 | 961 | 121 | 17 | 0 | 0 | 0 |
| | 2 | 3844 | 157 | 23 | 0 | 0 | 0 |
| | 4 | 7589 | 192 | 25 | 0 | 0 | 0 |
| | 6 | 50843 | 571 | 49 | 4 | 0 | 0 |
| | 12 | 70765 | 1615 | 204 | 36 | 4 | 0 |
| | 15 | 65410 | 4227 | 732 | 157 | 55 | 0 |

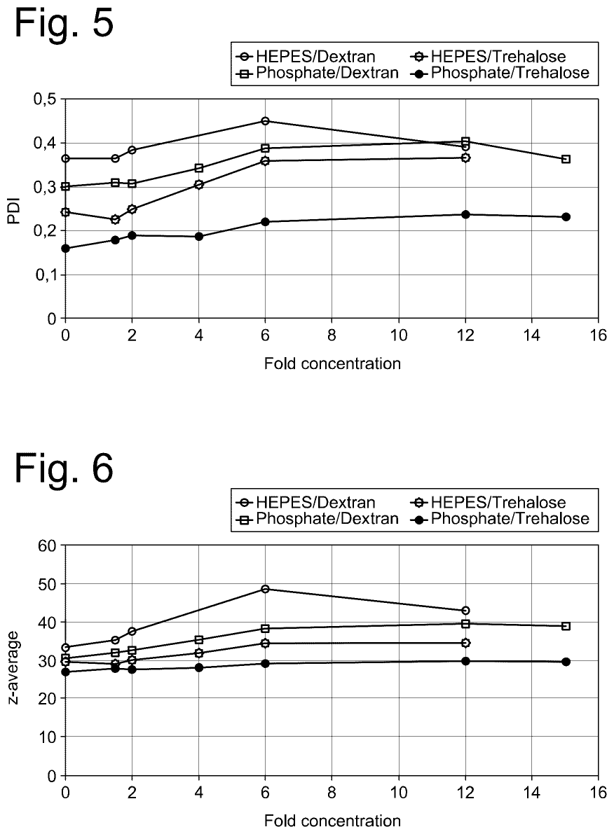

Polydispersity index (PDI) and z-average measurements by dynamic light scattering give information regarding the particle size, uniformity, and shape of the formulations under study. The results of PDI and z-average for the formulations are shown in FIGS. 5 and 6. PDI is a representation of the distribution of size populations within a given sample. The numerical value of PDI ranges from 0.0 (for a perfectly uniform sample concerning the particle size) to 1.0 (for a highly polydisperse sample with multiple particle size populations). FIG. 6 shows that all four formulations reach a plateau after a six-fold concentration in agreement with the GC and TP measurements above. Furthermore, the presence of trehalose causes the aggregates to be smaller than those in the presence of Dextran 6000. The same effect can be stated for the phosphate-containing formulations which induce smaller particles than the HEPES counterparts. The behaviour of the z-average is the same as that of the PDI, as described above.

Table 10 below shows the results of the stability test.

TABLE 10
Stability test as a function of the fold concentration.

| | HEPES/Trehalose | | | HEPES/Dextran | | | Phosphate/Trehalose | | | Phosphate/Dextran | | |
|---|---|---|---|---|---|---|---|---|---|---|---|---|
| Fold | | 24 hrs | | | 24 hrs | | | 24 hrs | | | 24 hrs | |
| Concentration | T₀ | 2-8 C. | F/T | T₀ | 2-8 C. | F/T | T₀ | 2-8 C. | F/T | T₀ | 2-8 C. | F/T |
| 4 | C | C | C | C | C | C | C | C | C | C | C | C |
| 6 | C | C | C | C | C | Co | C | C | C | C | C | C |
| 12 | C | T | C | C | Co | Co | C | C | C | C | C | Co |
| 15 | | | | | | | C | C | C | C | Co | Co |

C = Clear; Co = collapsed; T = turbid.

The overall observations are:
Highest concentration achievable 6E14 gc/mL (6-fold concentration)
Formulations in phosphate buffer show better visible particle profiles beyond 6E14 gc/mL
Phosphate/trehalose shows the narrowest particle size distribution

### Formulation Confirmation Study

Two formulations were proposed as a stability-enhancing for further tests and optimizations:
Formulation 1. Phosphate/Trehalose: Phosphate (Na/K) 10 mM; NaCl 82 mM; KCl 3 mM; Trehalose 3% (w/v); pH 7.3.
Formulation 2. Phosphate/Dextran: Phosphate (Na/K) 10 mM; NaCl 125 mM; KCl 3 mM; Dextran 6000 2.2% (w/v); pH 7.3.

The two formulations were filled in the final volume (1.2 mL) and container closure vials (2 ml borosilicate type I vials closed with a siliconized stopper and a flip-off seal). These vials were subject to accelerated studies at different stresses: F/T, shaking, and short-term development stability at 25 and 40° C. and one-year development stability (−80° C. and 2-8° C.) and comprised the following:
Freeze at −80° C. (at least 1 h) and thaw at RT (1 hour) for 3 cycles (for actual cycles length, look above in the study dates schedule).
Shaking for 24 hours at 200 rpm.
Accelerated stability: Storage at 25° C. for 2 weeks
Accelerated stability: Storage at 40° C. for 1 week.
Short-term development stability studies at −20° C. for 3 months
Long-term development stability studies at −80° C. (targeted DP storage temperature) and 2-8° C. for 1, 3, 6, 9, and 12 months
For all the above-mentioned conditions initial measurements (T=0) were carried out.
Additional time points were added after 1 F/T, after 1 week at 25° C., and after 2 weeks at 40° C., for Appearance and Visual inspection

The measurements followed were as follows:
Appearance/Visible particles (VI)
Sub-visible particles
Genome copies (gc); which correlates with concentration
tp: Total particles
ip: Infective particles
gc/ip: (genome copies/infective particles); which correlates with the number of active particles
tp/gc (total particles/genome copies); which correlates with purity
PDI by DLS (polydispersity index); it is expected to be lower than 0.1 as we were aiming to obtain mono-dispersed systems

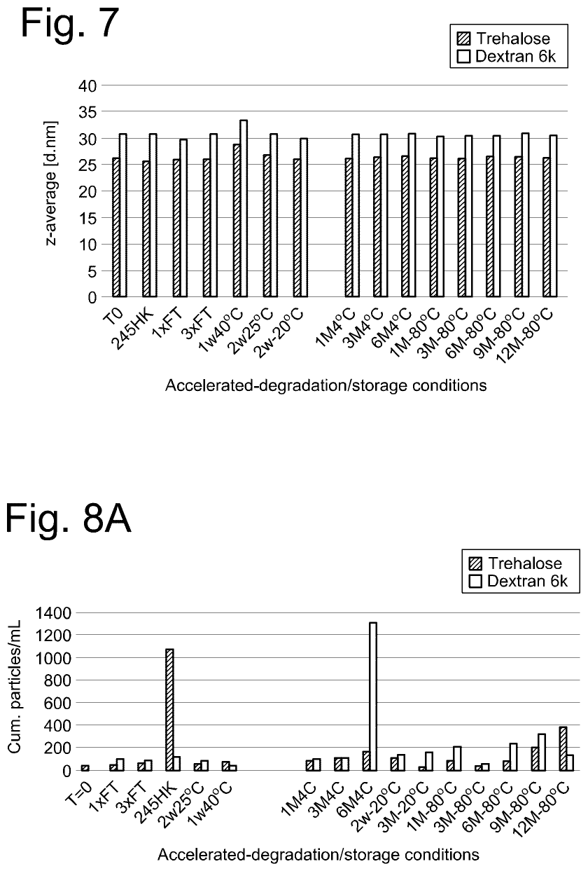

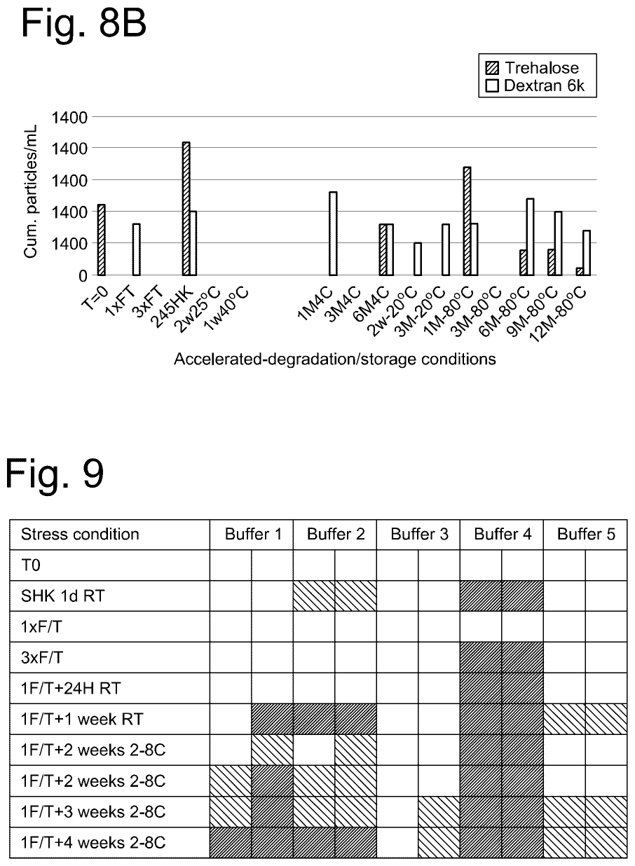

Full data is shown in Tables 11 to 13 below. Further data is also illustrated in FIGS. 7 and 8.

TABLE 11
Full data set for accelerated stressed condition studies.

| Formulation | | | | | | | |
|---|---|---|---|---|---|---|---|
| Buffer | Assay | Unit | t = 0 | SHK 24 hrs | 3x F/T | 40° C. 1 week | 25° C. 2 weeks |
| Formulation 1 | Genome Copies | gc/mL | 3.4E+14 | 2.5E+14 | 3.4E+14 | 4.0E+14 | 2.13e14 |
| | Total Particles | tp/mL | 3.6E+14 | 3.1E+14 | 3.1E+14 | 2.2E+14 | 2.8E+14 |
| | TP/GC | tp/gc | 1.1 | 1.2 | 0.9 | 0.6 | 1.3 |
| | Monomeric | % monomeric | 100 | 100 | 100 | 99.8 | 99.6 |
| | particle purity | Z-average | 26.47 | 25.94 | 26.37 | 29.02 | 27.08 |
| | | PdI | 0.111 | 0.087 | 0.113 | 0.215 | 0.180 |
| | | extra peaks | 1 | 0 | 0 | 2 | 2 |
| | Infectivity | ip/mL | 8.6E+12 | 7.8E+12 | 7.8E+12 | 6.7E+12 | N/D |
| | | gc/ip | 39.5 | 32.1 | 43.6 | 59.7 | N/D |
| Formulation 2 | Genome Copies | gc/mL | 4.2E+14 | 3.0E+14 | 3.1E+14 | 3.7E14 | 4.3E14 |
| | Total Particles | tp/mL | 4.9E+14 | 4.1E+14 | 2.1E+13 | 7.2E+13 | 3.8E+14 |
| | TP/GC | tp/gc | 1.2 | 1.4 | 0.1 | 0.2 | 0.9 |
| | Monomeric | % monomeric | 99.8 | 99.7 | 99.7 | 78.9 | 99.7 |
| | particle purity | Z-average | 30.69 | 30.66 | 30.59 | 33.75 | 30.87 |
| | | PdI | 0.218 | 0.232 | 0.236 | 0.31 | 0.242 |
| | | extra peaks | 2 | 2 | 1 | 1 | 1 |
| | Infectivity | ip/mL | 8.7E+12 | 7.7E+12 | 6.2E+12 | 9.2E+11 | 5.0E+12 |
| | | gc/ip | 48.3 | 38.4 | 49.2 | 402.2 | 86.0 |

TABLE 12
Full data set for long-term development stability studies at 2-8° C., −80° C. & −20° C.

| | | | | 4° C. | | | −80° C. | |
|---|---|---|---|---|---|---|---|---|
| Formulation | | | | 1 | 3 | 6 | 1 | 3 |
| Buffer | Assay | Unit | t = 0 | month | months | months | month | months |
| Formulation 1 | Genome | gc/mL | 3.4E+14 | 2.5E+14 | 2.7E+14 | 2.7E+14 | 2.4E+14 | 2.8E+14 |
| | Copies | | | | | | | |
| | Total | tp/mL | 3.6E+14 | 2.9E+14 | N/D | 2.6E+14 | 2.9E+14 | N/D |
| | Particles | | | | | | | |
| | TP/GC | tp/gc | 1.1 | 1.2 | N/D | 1.0 | 1.2 | N/D |
| | Monomeric | % monomeric | 100 | 100 | 99.9 | 100 | 100 | 100 |
| | particle | Z-average | 26.47 | 26.42 | 26.74 | 26.79 | 26.45 | 26.4 |
| | purity | PdI | 0.111 | 0.110 | 0.131 | 0.115 | 0.119 | 0.107 |
| | | extra peaks | 1 | 0 | 2 | 0 | 0 | 1 |
| | Infectivity | ip/mL | 8.6E+12 | 1.9E+13 | N/D | 7.0E+12 | 1.5E+13 | N/D |
| | | gc/ip | 39.5 | 13.2 | N/D | 38.6 | 16.0 | N/D |
| Formulation 2 | Genome | gc/mL | 4.2E+14 | 3.6E+14 | 3.5E+14 | 4.9E+14 | 3.2E+14 | 3.5E+14 |
| | Copies | | | | | | | |
| | Total | tp/mL | 4.9E+14 | 4.2E+14 | N/D | 3.5E+14 | 4.3E+14 | N/D |
| | Particles | | | | | | | |
| | TP/GC | tp/gc | 1.2 | 1.2 | N/D | 0.7 | 1.3 | N/D |
| | Monomeric | % monomeric | 99.8 | 99.7 | 99.8 | 99.7 | 99.6 | 99.8 |
| | particle | Z-average | 30.69 | 31.01 | 30.66 | 31.0 | 30.44 | 30.66 |
| | purity | PdI | 0.218 | 0.233 | 0.235 | 0.231 | 0.229 | 0.235 |
| | | extra peaks | 2 | 1 | 1 | 1.0 | 1 | 1 |
| | Infectivity | ip/mL | 8.7E+12 | 1.5E+13 | N/D | 8.2E+12 | 9.7E+12 | N/D |
| | | gc/ip | 48.3 | 24.0 | N/D | 59.8 | 33.0 | N/D |

| | | | −80° C. | | | −20° C. |
|---|---|---|---|---|---|---|
| Formulation | | | 6 | 9 | 12 | 3 |
| Buffer | Assay | Unit | months | months | months | months |
| Formulation 1 | Genome | gc/mL | 2.9E+14 | 3.0E+14 | 2.8E+14 | 2.7E+14 |
| | Copies | | | | | |
| | Total | tp/mL | 2.6E+14 | 2.6E+14 | 2.4E+14 | N/D |
| | Particles | | | | | |
| | TP/GC | tp/gc | 0.9 | 0.9 | 0.9 | N/D |
| | Monomeric | % monomeric | 100 | 100 | 100 | 100 |
| | particle | Z-average | 26.85 | 26.75 | 26.53 | 26.18 |
| | purity | PdI | 0.124 | 0.114 | 0.113 | 0.108 |
| | | extra peaks | 0 | 1 | 0 | 0 |
| | Infectivity | ip/mL | 7.5E+12 | 9.9E+12 | 6.2E+12 | N/D |
| | | gc/ip | 38.7 | 30.3 | 45.2 | N/D |
| Formulation 2 | Genome | gc/mL | 4.3E+14 | 4.0E+14 | 3.8E+14 | 4.2E+14 |
| | Copies | | | | | |
| | Total | tp/mL | 3.5E+14 | 3.4E+14 | 3.1E+14 | N/D |
| | Particles | | | | | |
| | TP/GC | tp/gc | 0.8 | 0.9 | 0.8 | N/D |
| | Monomeric | % monomeric | 99.8 | 99.5 | 99.7 | 99.8 |
| | particle | Z-average | 30.64 | 31.04 | 30.68 | 30.08 |
| | purity | PdI | 0.225 | 0.225 | 0.217 | 0.228 |
| | | extra peaks | 1.000 | 2.000 | 1.000 | 1 |
| | Infectivity | ip/mL | 7.2E+12 | 1.0E+13 | 9.1E+12 | N/D |
| | | gc/ip | 59.7 | 40.0 | 41.8 | N/D |

TABLE 13
Visual inspection results of the stress tests

| | Trehalose (Formulation 1) | | | | | | | | | Dextran 6K (Formulation 2) | | | | | | | | |
|---|---|---|---|---|---|---|---|---|---|---|---|---|---|---|---|---|---|---|
| stress | | 24 | 1 | 2 | 1 | 3 | 6 | 9 | | | 24 | 1 | 2 | 1 | 3 | 6 | 9 | |
| condition | F/T | hrs | w | w | M | M | M | M | 12 | F/T | hrs | w | w | M | M | M | M | 12 |
| 40 C. | | | Co | Co | | | | | | | | Co | | | | | | |
| 25 C. | | | | Co | Co | | | | | | | | Co | | | | | |
| 2-8 C. | | | | | Co | Co | Co | | | | | | | Co | Co | Co | | |
| −20 C. | | | | T | | T | | | | | | | Co | | Co | | | |
| ≤−65 | | | | | T | T | T | C | C | | | | | T | T | T | T | T |
| 1F/T | T | | | | | | | | | Co | | | | | | | | |
| 3F/T | T | | | | | | | | | Co | | | | | | | | |
| SHK | | Co | | | | | | | | | Co | | | | | | | |

C = Clear;
Co = collapsed;
T = turbid

The results confirm that the formulation containing trehalose is preferred to the one containing Dextran 6000. The strongest data supporting this conclusion is the visual inspection analysis, which shows that the formulations with trehalose show much less particles after F/T cycles or (long-term) storage at −80° C. Furthermore, the DLS measurements show that the PDI-value of the samples containing trehalose is almost half of the PDI value for samples containing Dextran 6000, meaning that the samples containing trehalose are more homogeneous than the ones containing Dextran 6000. The overall conclusion from the subvisible particle data is that trehalose shows much better results for the long term storage conditions.

The formulation: Phosphate (Na/K) 10 mM; NaCl 82 mM; KCl 3 mM; Trehalose 3% (W/V); pH 7.3 is recommended for further optimizations.

A further study was performed to select one of the following five different formulations, which contain different DP concentrations, MgCl₂ and/or PS-20 for the drug product. The previous early formulation report suggested a formulation with phosphate (Na/K) 10 mM; NaCl 82 mM; KCl 3 mM; Trehalose 3% w/v; pH 7.3 is recommended as the initial point for further optimizations.

The distinct DP materials were formulated as follows:
1. 3E14 gc/mL in trehalose/phosphate buffer;
2. 4E14 gc/mL in trehalose/phosphate buffer;
3. 3E14 gc/mL in trehalose/phosphate buffer+0.8 mM MgCl₂;
4. 3E14 gc/mL in trehalose/phosphate buffer+0.005% v/v PS-20;
5. 3E14 gc/mL in trehalose/phosphate buffer+0.01% v/v PS-20.

The trehalose/phosphate buffer comprises 9 mM phosphate buffer, 82 mM NaCl, 3 mM KCl and 3% w/v trehalose. These formulations were placed on stress studies as and the results are shown in Table 14 below.

TABLE 14
Results of the stress tests

| | | | | Time points | | | | |
|---|---|---|---|---|---|---|---|---|
| | | at T0 | | | | 1 D | 1 M | 1 W |
| | | gc/mL | tp/mL | Vial | T0 | SHK | 2-8 C. | 40 C. |
| Formulation | 3.0E+14 gc/mL | 2.50E+14 | 2.60E+14 | 1 | C | T | Co | Co |
| | | | | 2 | C | T | C | Co |
| | | | | 3 | C | T | C | |
| | 3.0E+14 gc/mL | 3.10E+14 | 2.70E+14 | 1 | C | C | C | C |
| | 0.005% PS20 | | | 2 | C | C | C | C |
| | | | | 3 | C | C | C | |
| | 3.0E+14 gc/mL | 2.90E+14 | 2.70E+14 | 1 | C | C | C | C |
| | 0.01% PS20 | | | 2 | C | C | C | C |
| | | | | 3 | C | C | C | |
| | 3.0E+14 gc/mL | 2.70E+14 | 2.70E+14 | 1 | C | T | Co | Co |
| | 0.8M MgCl2 | | | 2 | C | T | Co | Co |
| | | | | 3 | C | T | C | |
| | 4.0E+14 gc/mL | 3.40E+14 | 3.60E+14 | 1 | C | T | Co | Co |
| | | | | 2 | C | T | C | Co |
| | | | | 3 | C | T | | |

C = Clear; Co = collapsed; T = turbid.

The results shown above confirm that the addition of PS-20 (in particular at 0.01% v/v) has a positive effect on the stability of the formulation.

### Conclusion

Plate-based screening studies highlighted stabilizing effect of NaCl and neutral to slightly alkaline pH
Studies show stabilizing effect of trehalose and dextran 6k in either phosphate or HEPES buffer
Phosphate+trehalose/dextran 6k supports concentrations up to 6E14 gc/mL
Phosphate+trehalose shows superior visible particle profile
Formulation optimization through the addition of 0.01% v/v PS-20 improves visual appearance & greatly reduces subvisible particles
Formulation has been optimized for frozen storage at ≤−65° C.

One particular embodiment of the present invention comprises the compositions shown in Table 15 below, in addition to the adeno-associated viral vector with a transgene encoding a microRNA.

TABLE 15
Buffers analysed

| | | Buffer (2) | | | Buffer (5) |
|---|---|---|---|---|---|
| | Buffer (1) | PBS + 0.001% | Buffer (3) | Buffer (4) | Zolgensma |
| Parameter | Zolgensma | Poloxamer 188 | AMT-150 FB | AMT-130 FB | modified |
| Buffer/pH | 20 mM Tris | 10 mM | 9 mM | 20 mM Tris | 20 mM Tris |
| | pH 8.0 | phosphate | phosphate | pH 7.5 | pH 8.0 |
| | | pH 7.4 | pH 7.3 | | |
| Tonicifier | 200 mM NaCl | 137 mM NaCl, | 87 mM NaCl, | 135 mM NaCl | 135 mM NaCl |
| | | 2.7 mM KCl | 3 mM KCl | | |
| Cryoprotectant | X | X | 3% | 0.1% Mannitol | X |
| | | | Trehalose•2H₂O | 0.1% HP-b-BCD | |
| Surfactant | 0.005% | 0.001% | 0.01% PS20 | X | 0.005% |
| | Poloxamer 188 | Poloxamer 188 | | | Poloxamer 188 |
| Other ingredients | 1 mM MgCl2 | X | X | 2.5 mM His | 1 mM MgCl2 |
| Theoretical | 423 | 309 | 286 | 299 | 293 |
| Osmo (mOsm/L) | | | | | |

### Example 2—Formulation Study AAV9

The drug product (DP) used in this example was AAV9 with a transgene encoding a luciferase reporter at a concentration of approximately 3E13 gc/mL (see Table 19).

The composition of the five formulation buffers used in this study are listed in Table 15. All five buffers were prepared in a 4 L volume and they were filtered using a 0.22 μm PES filter. Poloxamer 188 and PS20 surfactants were added freshly to the corresponding formulation buffers on the date of the buffer exchange process.

30 mL of AAV9 formulation per buffer condition was exchanged using dialysis. Three cycles of buffer exchange (1 L per cycle) were performed for 16 hours at 2-8° C.

AAV9 collected volumes after buffer exchange process were filtered using a 0.22 μm PVDF syringe filter. AAV9 DP was filled in 2 mL type I glass vials with a fill volume of 1.2 mL DP. Table 16 shows the total number of filled DP vials per collected AAV9 volume in each formulation buffer.

TABLE 16
Number of AAV9 DP filled vials per each formulation buffer

| | Volume after buffer | | Number of filled |
|---|---|---|---|
| DP description | exchange and filtration | | (1.2 mL) glass vials |
| AAV9 in formulation | 31 | mL | 25 |
| buffer 1 | | | |
| AAV9 in formulation | 26.5 | mL | 21 |
| buffer 2 | | | |
| AAV9 in formulation | 26 | mL | 21 |
| buffer 3 | | | |
| AAV9 in formulation | 31.5 | mL | 25 |
| buffer 4 | | | |
| AAV9 in formulation | 27 | mL | 23 |

buffer 5

All filled DP vials were visually inspected. DP vials that passed visual inspections were placed under different stress conditions as listed in Table 17 and routed for analysis as described in Table 18.

TABLE 17
Number of AAV9 DP vials per each stress condition

| | Formulation | | | | |
|---|---|---|---|---|---|
| | AAV9 | AAV9 | AAV9 | AAV9 | AAV9 |
| Stress Conditions | buffer 1 | buffer 2 | buffer 3 | buffer 4 | buffer 5 |
| T = 0 | 2 | 2 | 2 | 2 | 2 |
| SHK, 1 Day | 2 | 2 | 2 | 2 | 2 |
| (200 rpm, RT) | | | | | |
| 1 × FT | 2 | 2 | 2 | 2 | 2 |
| 1 × FT + 24 hrs at RT | 2 | 2 | 2 | 2 | 2 |
| 3 × FT | 2 | 2 | 2 | 2 | 2 |
| 1 × FT + 1 W at RT | 2 | 2 | 2 | 2 | 2 |
| 1 × FT + 4 W at 2-8° C. | 2 | 2 | 2 | 2 | 2 |
| Total number of DP | 14 | 14 | 14 | 14 | 14 |
| vials | | | | | |

TABLE 18
Overview of the analytical methods

| | Assay | Volume (mL) per |
|---|---|---|
| Quality attributes/Method | format | buffer condition |
| Genome copies/Quantitative | 2 × 3 | 3 × 0.25 |
| Polymerase Chain Reaction (QPCR) | | |
| pH/Potentiometry | 1 × 1 | 1 × 0.7 |
| Osmolality/Freezing point | 1 × 1 | 1 × 0.25 |
| depression | | |
| Visible particulates/Visual | 200% | 2 × 1.2 |
| inspection | | |
| Vis and Sub-visible particulates/ | 1 × 2 | 2 × 0.025 |
| Background membrane imaging | | |
| Subvisible particulates/Light | 2 × 1 | 2 × 1.2 |
| obscuration | | |
| Purity, % monomer/HPL-SEC | 1 × 2 | 1 × 0.25 |
| Hydrodynamic radius and | 1 × 3 | 3 × 0.03 |
| polydispersity index/Dynamic Light | | |
| Scattering (DLS) | | |
| Temperature of unfolding/Nano | 1 × 2 | 2 × 0.01 |
| Differential Scanning Fluorimetry | | |
| (nanoDSF) | | |
| DNA, Protein absorbance (UV260, | 1 × 2 | 2 × 0.1 |
| UV280) and Intrinsic Fluorescence | | |
| (F350 nm)/Spectroscopy | | |
| Transduction efficiency/Dual | 2 × 3 | 2 × 0.1 |
| Reporter Luciferase Assay | | |

### Results

### Genome Copies

Genome copy (GC) measures the amount of AAV particles that have the vector DNA packaged within the particle. GC concentration is determined by quantitative POR. GC assay was performed for T=0 (after buffer exchange), SHK (1 Day, 200 rpm), 1 FT+7 Days and 1 FT+4 W at 2-8° C. stress conditions. The averaged GC triplicates for two vials per formulation buffer at T=0, are shown in Table 19.

TABLE 19
Genome copies of AAV9 DP in different
formulation buffers after DP fill

| Description | Genome copies (gc/mL) |
|---|---|
| AAV9 Buffer 1 | 3.17E+13 |
| AAV9 Buffer 2 | 2.98E+13 |
| AAV9 Buffer 3 | 4.00E+13 |
| AAV9 Buffer 4 | 3.27E+13 |
| AAV9 Buffer 5 | 2.67E+13 |

The AAV9 GC was around 3E13 gc/ml among all tested formulations taking in consideration the 20% GC assay variation. A slightly higher concentration of AAV9, 4E13 gc/ml, was measured in formulation buffer 3. No observable loss of GC was detected in either of the tested samples under the different stress conditions.

### pH and Osmolality

pH and osmolality data are close to the theoretical values for each formulation buffer (see Table 20), which indicates that buffer exchange was performed correctly.

TABLE 20
pH and osmolality of AAV9 DP in different formulation buffers

| | | | Osmolarity | Osmolality |
|---|---|---|---|---|
| | pH | | Theoretical | Measured |
| Description | targeted | measured | mOsm/L | mOsm/kg |
| AAV9 NF starting | 8.3 | 8.5 | 600 | 687 |
| material | | | | |
| AAV9 Buffer 1 | 8.0 | 7.9 | 423 | 398.0 |
| AAV9 Buffer 2 | 7.4 | 7.3 | 309 | 282.5 |
| AAV9 Buffer 3 | 7.3 | 7.7 | 286 | 268.5 |
| AAV9 Buffer 4 | 7.5 | 7.6 | 299 | 287.5 |
| AAV9 Buffer 5 | 8.0 | 7.8 | 293 | 278.0 |

Before buffer exchange, measured AAV9 buffer osmolality was 687 mOsm/kg and pH 8.5, buffer composition being 0.1M Glycine, 0.5M Tris, pH 8.3. After buffer exchange, all formulations were close to the targeted pH and theoretical osmolarity. With the exception of buffer 4, Zolgesma's formulation buffer an hypertonic formulation, all other formulations are isotonic formulations.

### Visual and Sub-Visible Particle Analysis

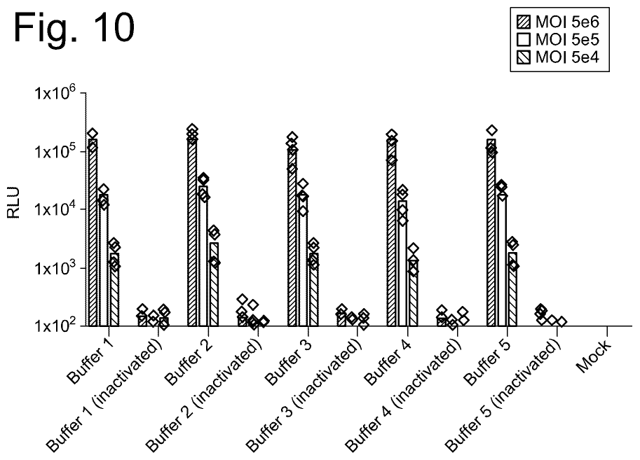

Visual inspection acceptance criteria was aligned to the USP <790> specification and defined as colourless liquid, clear, free of visible particles. Visual inspection was performed against a white and black background. All AAV9 DP vials were visually inspected after fill and after applied stresses. DP vials containing extrinsic particles after filling and those with low filling volumes were rejected. The results are shown in Table 21 and FIG. 9.

TABLE 21
Visual inspection results after DP Fill and finish, T = 0

| | | | | Description | No. of | |
|---|---|---|---|---|---|---|
| Type of | Total | | Color of | of | rejected | Remaining |
| formulation | Vials | Clarity | Liquid | Particulates | vials | vials |
| AAV9 Buffer 1 | 25 | Clear | Colorless | One vial with | 1 | 24 |
| | | | | particle | | |
| AAV9 Buffer 2 | 22 | Clear | Colorless | One vial with | 1 | 21 |
| | | | | low volume | | |
| AAV9 Buffer 3 | 21 | Clear | Colorless | One vial with | 1 | 20 |
| | | | | particle | | |
| AAV9 Buffer 4 | 26 | Clear | Colorless | One vial with | 1 | 25 |
| | | | | low volume | | |
| AAV9 Buffer 5 | 23 | Clear | Colorless | One vial with | 1 | 22 |
| | | | | low volume | | |

Two vials per formulation buffer and stress condition were visually inspected. AAV9 DP was most stable in formulation buffer 3. In this formulation, AAV9 DP remained free of particles after agitation for one day at RT, after three freeze/thaw cycles, and after one week after thawing when stored at RT or two weeks after thawing when stored at 2-8° C. respectively (FIG. 9). Two vials per stress condition and formulation buffer were analyzed for the subvisible particles using the light obscuration technique AccuSizer FX.

Under all stress conditions, subvisible particles remained within the acceptance criteria according to USP<787>: 6000 particles/mL above 10 μm and 600 particles/mL above 25 μm. A similar aggregation profile was observed for all tested formulation buffers in the 0.3-1 μm region. AAV9 aggregation was accelerated upon freeze-thaw stresses, while agitation stress condition had less of an impact on aggregation.

It is generally observed that the number of subvisible particles larger than 2 μm of AAV9 in formulation buffer 3 is less when compared to the other formulations under all applied stress conditions.

### Background Membrane Imaging (BMI)

Particles from 2 μm to 4 μm were visualized with Aura system that uses BMI technology, a form of automated membrane microscopy that enables particle counting, sizing, and morphology in a 96 well format. AAV9 DP in formulation buffer 4 after 1 FT+4 weeks at 2-8° C. contains the highest number of particles >2 μm when compared to the other formulations in agreement with the results from visual inspection related to visible particles and sub-visible particle analysis by light obscuration technique.

AAV9 Transduction into Target Cells

Potential effect of the different formulations on AAV9 transduction efficiency has been evaluated in vitro in a cellular assay. For each buffer condition, AAV9 transduction efficiency was tested in human hepatoma-derived carcinoma (Huh7) cells. Huh7 cells were chosen as an in vitro model cell line based on previously published data by Pietersz et al., 2021 (Pietersz K L, Plessis F D, Pouw S M, Liefhebber J M, van Deventer S J, Martens G J M, Konstantinova P S, Blits B. PhP.B Enhanced Adeno-Associated Virus Mediated-Expression Following Systemic Delivery or Direct Brain Administration. Front Bioeng Biotechnol. 2021 Aug. 3; 9:679483. doi: 10.3389/fbioe.2021.679483. PMID: 34414171: PMCID: PMC8370029).

Huh7 cells were co-transduced with the differently formulated AAV9 DPs and wild-type Adeno5, Ad5 ARM adenovirus, at three different AAV9 doses (GC/cell) also known as multiplicity of infections (MOI). In addition to the untreated AAV9 in the different formulation buffers, 50 μL of AAV9 per buffer condition was heat-inactivated by incubation at 95° C. for 10 min as a negative control for each condition. The ready out was performed using Dual-Luciferase Reporter Assay System.

As shown in FIG. 10, from the luciferase activity in the cell lysates of Huh-7 cells transduced with AAV9, it can be concluded that the different buffer formulations did not significantly impact the transduction efficiency of AAV9 regardless of the MOI used.

### Conclusion

A platform formulation fit study was carried out to select the most suitable formulation from already developed formulations for brain administration in which AAV9-luciferase proves to be most stable under selected stress conditions.

Overall, AAV9 DP was most stable in formulation buffer 3 under applied stress conditions. No visible particles and less sub-visible particles above 2 μm were observed in formulation buffer 3 when compared to the other selected formulations upon all applied stress conditions. The subvisible particles remained within the acceptance criteria according to USP<787>: 6000 particles/mL above 10 μm and 600 particles/mL above 25 μm. The other quality attributes, genome copies, SEC purity (% monomer), hydrodynamic radius and polydispersity index and temperature of unfolding, showed no significant changes in response to the applied stresses when compared to T=0. In addition, AAV9 transduction into the target cells was not impacted by the formulation buffers regardless of multiplicity of infection used.

Therefore buffer 3 is considered the most suitable formulation for preserving the quality of AAV9-luciferase product and should be considered for subsequent studies. Furthermore, this formulation is an isotonic formulation, with a similar tonicity and pH as cerebrospinal fluid: 280-310 mOsm/kg and pH 7.3-7.4 (Salvador L, Valero R, Carrero E, Caral L, Fernández S, Marin J L, Ferrer E, Fábregas N. Cerebrospinal fluid composition modifications after neuroendoscopic procedures. Minim Invasive Neurosurg. 2007 February; 50(1):51-5. doi: 10.1055/s-2007-973823. PMID: 17546545).

TABLE 22
Formulation of one particular embodiment of the present invention
(in addition to the adeno-associated viral vector)

| Formulation buffer | Cryoprotectant | Tonicity agent | Surfactant |
|---|---|---|---|
| 10 mM Na/K-phosphate | 3% (w/v) | 3 mM KCl | 0.01% (v/v) |
| | trehalose | 87 mM NaCl | PS-20 |

TABLE 23
Formulation of one particular embodiment of the present invention
(in addition to the adeno-associated viral vector)

| Formulation buffer | Cryoprotectant | Tonicity agent | Surfactant |
|---|---|---|---|
| 10 mM Na/K-phosphate | 3% (w/v) | 3 mM KCl | 0.01% (v/v) |
| | trehalose | 82 mM NaCl | PS-20 |

1. An isotonic pharmaceutical formulation comprising:
a buffer
an adeno-associated viral vector with a transgene encoding a microRNA;
a tonicity agent;
a cryoprotectant; and
a surfactant.

2. The formulation of claim 1, wherein the formulation has an osmolarity of from 250 to 330 mOsm/kg and a pH value of from 5 to 8.

3. The formulation according to claim 1, wherein the formulation is substantially free of visible particles.

4. The formulation according to claim 1, wherein the adeno-associated viral vector with a transgene encoding microRNA targets ATXN mRNA, for example, ATXN3 mRNA.

5. The formulation according to claim 1, wherein the adeno-associated viral vector comprises an AAV2 serotype, an AAV5 serotype, an AAV9 serotype, a hybrid AAV serotype, or a combination thereof.

6. The formulation according to claim 1, wherein the concentration of the adeno-associated viral vector is at least 1E13 gc/ml, such as at least 3E13 gc/ml.

7. The formulation according to claim 1, wherein the cryoprotectant is a carbohydrate selected from the group consisting of trehalose, sucrose, dextrose, dextran, and combinations thereof; and present in an amount of up to 8% w/v.

8. The formulation according to claim 1, wherein the tonicity agent is selected from the group consisting of NaCl, KCl, MgCl₂, CaCl₂, and combinations thereof; and is present in an amount of from 5 mM to 150 mM.

9. The formulation according to claim 1, wherein the surfactant is a non-ionic surfactant; and is present in an amount of from 0.001% v/v to 0.1% v/v.

10. The formulation according to claim 1, wherein the buffer is selected from the group consisting of an artificial cerebrospinal fluid (aCSF), acetate buffer, citrate buffer, phosphate buffer, HEPES, Tris, and combinations thereof.

11. The formulation according to claim 1, wherein the formulation remains stable at room temperature (15° C.-25° C.) without substantial formation of aggregates or agglomerates for at least 12 hours after thawing.

12.-14. (canceled)

15. A method for treating Spinocerebellar Ataxia (SCA) in a patient, the method comprising administering the formulation according to claim 1.

16. The method according to claim 15, wherein the formulation is administered by intra-CSF (cerebral spinal fluid) delivery.

17. The method according to claim 15, wherein the formulation is administered to a patient's cisterna magna.
